(19) United States
(12) Patent Application Publication
(10) Pub. No.: US 2026/0015587 A1
(43) Pub. Date: Jan. 15, 2026

(54) COMPOSITIONS AND METHODS FOR GROWING ESOPHAGEAL CELLS AND RELATED USES THEREOF

(71) Applicant: The Regents of the University of Michigan, Ann Arbor, MI (US)

(72) Inventors: Jason Spence, Ann Arbor, MI (US); Daysha Ferrer-Torres, Ann Arbor, MI (US)

(21) Appl. No.: 18/994,414

(22) PCT Filed: Aug. 21, 2023
(86) PCT No.: PCT/US2023/030670

§ 371 Date: Jan. 14, 2025

Related U.S. Application Data
(60) Provisional application No. 63/399,891, filed on Aug. 22, 2022.

(51) Int. Cl. C12N 5/071

(52) U.S. Cl. CPC C12N 5/0679; C12N 2501/11; C12N 2501/15; C12N 2501/155; C12N 2501/415; C12N 2501/727; C12N 2501/999; C12N 2502/13; C12N 2513/00; C12N 2533/90

(57) ABSTRACT

Improved techniques for maintaining long-term cultures of the healthy and diseased human esophagus established in 2-dimension (2D) and 3-dimension is also needed. The invention disclosed herein generally relates to methods and systems for culturing and expanding esophageal cells with a growth media comprising a rho-kinase inhibitor, a WNT-activator/agonist, EGF, an inhibitor of TGF-BETA signaling, an inhibitor of BMP signaling, and hydrocortisone.

### STATEMENT OF RELATED APPLICATIONS

This application claims the benefit of U.S. Provisional Patent Application No. 63/399,891, filed Aug. 22, 2022, the contents of each of which are incorporated by reference herein.

### FIELD OF THE INVENTION

The invention disclosed herein generally relates to methods and systems for culturing and expanding esophageal cells with a growth media comprising a rho-kinase inhibitor, a WNT-activator/agonist, EGF, an inhibitor of TGFβ signaling, an inhibitor of BMP signaling, and hydrocortisone.

### Introduction

The esophagus connects the upper pharynx with the stomach and is lined by a stratified squamous epithelium (Rosekrans et al. 2015). In early development, the esophagus and trachea develop from a common gut tube, which separates to give rise to the esophagus and the respiratory organs during the first trimester of the human fetal development (Rankin et al. 2016; Ernst et al. 2019). The esophagus is specified on the dorsal side of the common gut tube, with the lung being specified on the ventral side (Kuwahara et al. 2020). At this early time, the esophagus is organized as a pseudostratified epithelium that matures into a fully stratified epithelium (Ernst et al. 2019). Failure to achieve proper specification and separation can lead to different developmental defects, such as esophageal atresia or tracheoesophageal fistula (Houben, C. H. and Curry 2008).

The stratified epithelium of the esophagus is maintained throughout adulthood. Histologic and more recent single-cell characterization of the esophagus has identified several distinct epithelial cell types/states (Madissoon et al. 2019; Bogte et al. 2021). Understanding, describing, and characterizing the normal/healthy esophagus during homeostasis is an essential first step to understanding injury and repair, disease states, and disease progression. In vitro model systems are critical to studying human health and disease. The adult esophagus is prone to many diseases, including eosinophilic esophagitis (Blevins et al. 2018), metaplasia (Barrett's esophagus) due to reflux and inflammation, esophageal squamous cancer (Kim et al. 2017), and esophageal adenocarcinoma (Saraggi et al. 2016). Genetic variation attributed to ancestry and racial background has been linked with the esophageal disease despite risk factors being equal across populations (S. J. Spechler et al. 2011; S. Spechler et al. 2002; Thrift and El-Serag 2016; Hashem B. and El-Serag et al. 2004). For example, there is a higher incidence of esophageal adenocarcinoma in the Caucasian/European American (EA) population when compared to populations of African or African descent (AA) (El-Serag, HB and Sonnenberg 1997; Rastogi et al. 2008; Fleischer et al. 2008; H. B. El-Serag et al. 2014; Arnold et al. 2017; Then et al. 2020).

An improved understanding of esophageal development is needed. Improved techniques for maintaining long-term cultures of the healthy and diseased human esophagus established in 2-dimension (2D) and 3-dimension is also needed.

The present invention addresses these needs.

### SUMMARY OF THE INVENTION

Experiments conducted during the development of embodiments for the present invention were conducted, for example, to leverage access to patient biopsy specimens to create an atlas of cell types within the healthy human esophagus and to use this atlas to benchmark in vitro models. The experimental results described herein adds to prior studies by (Mou et al. 2016; Yamamoto et al. 2016; Kasagi et al. 2018; Liu et al. 2017; Trisno et al. 2018; Zhang et al. 2018), providing a detailed in vivo single-cell atlas that is used to benchmark in vitro grown human esophagus. Single-cell RNA-seq data obtained from healthy esophagus biopsies identified four molecularly distinct epithelial cell types corresponding to different domains within the epithelium, which were validated by immunofluorescence. Within the basal cell compartment, where stem cells reside, such experiments resulted in the identification of previously known (Bogte et al. 2021) and new markers (i.e., COL17A1⁺, CAV1⁺, CAV2⁺). Adjacent to the basal cell domain was the suprabasal zone, which can be divided into three distinct domains based on protein markers: an LY6D⁺ epibasal domain, which corresponds to the domain with high proliferation (KI67⁺), a KRT4⁺ mid differentiation zone, and a CRNN⁺ luminal zone of terminally differentiated cells.

To develop a robust method to maintain long-term cultures of the healthy and diseased human esophagus established in 2-dimension (2D) in vitro, such experiments resulted in the unification of different methods reported for the culture of rodent and human esophagus (DeWard, Cramer, and Lagasse 2014; Bogte et al. 2021; Madissoon et al. 2019; Mou et al. 2016; Liu et al. 2017; Yamamoto et al. 2016; Kasagi et al. 2018). It was demonstrated that the method described herein is suited for 2D in vitro culture of the adult and developing human esophagus and esophagus tissue from healthy and diseased states. Using these methods, a diverse growing biobank (currently n=55 cell lines) was developed from individuals self-identified as European American, African American/Black, Asian, or of Hispanic descent. By comparing the in vivo reference atlas to the in vitro scRNA-seq data, it was found that 2D in vitro grown cultures possessed an abundance of cells transcriptionally like basal-epibasal cells. These results demonstrate that cell density determines the balance of proliferation and differentiation within the culture. Basal cells maintained in two-dimensional culture can also be used to grow three-dimensional (3D) organoids. Taken together, the experiments described herein provide detailed methods for the long-term culture of the human esophagus in vitro, provide an atlas of the adult human esophagus, and use scRNA-seq data as a roadmap to benchmark in vitro cultured cells providing a detailed foundation for pursuing human esophagus research.

Accordingly, the invention disclosed herein generally relates to methods and systems for culturing and expanding esophageal cells with a growth media comprising a rho-kinase inhibitor, a WNT-activator/agonist, EGF, an inhibitor of TGFβ signaling, an inhibitor of BMP signaling, and hydrocortisone.

In certain embodiments, the present invention provides methods comprising culturing in vitro obtained esophageal cells with a growth media comprising a rho-kinase inhibitor, a WNT-activator/agonist, EGF, an inhibitor of TGFβ signaling, an inhibitor of BMP signaling, and hydrocortisone, wherein the culturing results in expansion of the obtained esophageal cells.

In certain embodiments, the present invention provides methods of generating stratified esophageal epithelium, comprising culturing obtained esophageal cells in vitro, wherein the culturing results in expansion of the obtained esophageal cells into stratified esophageal epithelium, wherein the culturing comprises a growth medium comprising hydrocortisone, epidermal growth factor (EGF), a rho-kinase inhibiting agent, a Wnt signaling pathway activating agent, a TGFβ inhibiting agent, and a BMP inhibiting agent.

Such methods are not limited to a particular Wnt signaling pathway activating agent within the growth medium. In some embodiments, the Wnt signaling pathway activating agent is a small molecule or agonist that activates the Wnt signaling pathway. In some embodiments, the small molecule or agonist that activates the Wnt signaling pathway is CHIR99021. In some embodiments, activating the Wnt signaling pathway occurs through culturing the esophageal cells with one or more molecules configured to activate a Wnt protein, wherein the Wnt protein is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, activating the Wnt signaling pathway comprises culturing the esophageal cells with a small molecule or other agonist that stimulates Wnt signaling. In some embodiments, the Wnt agonist is CHIR99021.

Such methods are not limited to a particular rho-kinase inhibiting agent within the growth medium. In some embodiments, the agent is a small molecule or antagonist that inhibits rho-kinase. In some embodiments, the rho-kinase inhibiting agent is Y-276327.

Such methods are not limited to a particular BMP inhibiting agent within the growth medium. In some embodiments, the BMP inhibiting agent is a small molecule or antagonist that inhibits the BMP signaling pathway. In some embodiments, the small molecule or antagonist that inhibits the BMP signaling pathway is Noggin.

Such methods are not limited to a particular TGFβ inhibiting agent within the growth medium. In some embodiments, the TGFβ inhibiting agent is a small molecule or antagonist that inhibits the TGFβ signaling pathway. In some embodiments, the small molecule or antagonist that inhibits the TGFβ signaling pathway is A-8301. In some embodiments, the small molecule or antagonist that inhibits the TGFβ signaling pathway is SB431542.

In some embodiments, the growth medium comprises hydrocortisone, EGF, Y-276327, CHIR99021, A-8301, and noggin.

In some embodiments, the expanded esophageal cells possess esophageal stem cells and differentiated esophageal cells. In some embodiments, the expanded esophageal cells are capable of cryopreservation. In some embodiments, the cryopreserved expanded esophageal cells can be thawed for purposes of generation of esophageal cell lines (e.g., 2-dimensional). In some embodiments, the cryopreserved expanded esophageal cells can be thawed for purposes of generation of esophageal organoid tissue (e.g., 3-dimensional).

In some embodiments, the expanded esophageal cells are stratified esophageal epithelium cells. In some embodiments, the stratified esophageal epithelium is 2-dimensional stratified esophageal epithelium. In some embodiments, the stratified esophageal epithelium is 3-dimensional stratified esophageal epithelium (e.g., 3-dimensional esophageal organoid tissue).

In some embodiments, the culturing further comprises seeding the obtained esophageal cells onto sub-lethally irradiated feeder cells and the growth media. In some embodiments, the sub-lethally irradiated feeder cells are 3T3-J2i sub-lethally irradiated feeder cells.

In some embodiments, the culturing further comprises seeding the obtained esophageal cells onto solubilized basement membrane matrix cells and the growth media. In some embodiments, the solubilized basement membrane matrix cells are matrigel cells.

Such embodiments are not limited to utilizing a specific amount of growth medium within the culturing step.

In some embodiments, the amount of growth medium utilized in the culturing step is approximately 2 ml within a 9.8 cm² well, which results in generation of 2-dimensional stratified esophageal epithelium.

In some embodiments, the amount of growth medium utilized in the culturing step is approximately 4 ml within a 9.8 cm² well, which results in generation of 3-dimensional stratified esophageal epithelium (e.g., 3-dimensional esophageal organoid tissue).

In some embodiments, the culturing of obtained esophageal tissue with the growth medium occurs over a specified temporal period. In some embodiments, the culturing of obtained esophageal tissue with the growth medium occurs for 6 or more hours; 12 or more hours; 18 or more hours; 24 or more hours; 36 or more hours; 48 or more hours; 60 or more hours; 72 or more hours; 84 or more hours; 96 or more hours; 120 or more hours; 150 or more hours; 180 or more hours; or 240 or more hours.

In some embodiments, the obtained esophageal cells are cultured with the growth medium at a concentration of ng/ml or higher, 20 ng/ml or higher; 50 ng/ml or higher; 75 ng/ml or higher; 100 ng/ml or higher; 120 ng/ml or higher; 150 ng/ml or higher, 200 ng/ml or higher; 500 ng/ml or higher; 1,000 ng/ml or higher; 1,200 ng/ml or higher, 1,500 ng/ml or higher; 2,000 ng/ml or higher, 5,000 ng/ml or higher, 7,000 ng/ml or higher, 10,000 ng/ml or higher; or 15,000 ng/ml or higher. In some embodiments, concentration of the growth medium is maintained at a constant level throughout the culturing. In other embodiments, concentration of the growth medium is varied during the course of the culturing.

Such embodiments are not limited to a particular type or kind of obtained esophageal cells. In some embodiments, the obtained esophageal cells are comprised within a biopsy sample from a subject (e.g., a human subject). In some embodiments, the obtained esophageal cells are comprised within a surgical specimen sample from a subject (e.g., a human subject). In some embodiments, the obtained esophageal cells are fetal esophageal cells. In some embodiments, the obtained esophageal cells are adult esophageal cells. In some embodiments, the obtained esophageal cells are esophageal stem cells. In some embodiments, the obtained esophageal cells are differentiated esophageal cells.

In some embodiments, prior to the culturing step the obtained esophageal cells are minced or enzymatically digested.

In certain embodiments, the present invention provides compositions comprising or consisting of esophageal cells expanded with the methods described herein.

In certain embodiments, the present invention provides kits comprising or consisting of stratified esophageal epithelium, 2-dimensional stratified esophageal epithelium, and/or 3-dimensional stratified esophageal epithelium (e.g., 3-dimensional esophageal organoid tissue) produced in vitro from the described methods.

### BRIEF DESCRIPTION OF DRAWINGS

FIG. 1: Human esophageal biopsies are characterized by scRNAseq. (A) Patient biopsies (n=2) were sequenced on the day of biopsy (day 0), and a total of 9,039 cells were included in the analysis after filtering, with 3,897 genes expressed per cell. Louvain clustering predicts seven molecularly distinct clusters (each cluster was determined using the top 200 genes differentially expressed in each cluster (P<0.01). (B) Individual patient samples have a similar distribution of cells to each cluster. x denotes the average contribution of both samples to the clusters. (C-D) Identification of epithelial cells (CDH1+) and lamina propria/mesenchymal cell types (VIM+) (arrows highlight papillae zones known to be present in the mature adult esophagus). (E-F) Dot plots of the top 5 genes expressed in each cluster (E) and feature plots of selected marker genes (F) were used to annotate each cluster. (G) Protein expression of top markers identified using scRNA-seq for the different epithelial zones of the esophagus (data courtesy of the Human Protein Atlas) (Uhlen et al. 2015). Scale bar represents 100 μm (20×).

FIG. 2: Identification of distinct molecular domains within the esophageal epithelium. (A) CDH1⁺ epithelial cells were sub-clustered from scRNA-seq data from normal match tissue esophageal biopsies [(n=2 biopsies/patients) (Pt #1:45 yr male, Pt #2: 63 yr female)] with a total of 7796 cells were analyzed after filtering, and 2651 genes per cell. Louvain clustering was used to predict clusters and visualized using UMAP. (B) Distribution of patient cells to each cluster. x denotes the average contribution of both samples to the clusters. (C) Dot-plots of the top 5 genes expressed in each cluster and annotations for each cluster based on top genes and known genes (see also Table S2). (D) Feature plots of top marker genes expressed in each cluster, with CAV1/COL17A1 for Cluster 2 (basal), KI67 for Cluster 1 (proliferative), LY6D, and KRT4 for Cluster 0 (epibasal/suprabasal), and CRNN for Cluster 3 (luminal). (E) Representative immunofluorescent images in the adult human esophagus validating expression of genes identified by scRNA-seq. The markers localize COL17A1, CAV1, and CAV2 (see FIG. 3) with the highest expression at the basal zone, KI67 marks proliferative cells at the basal-epibasal zone, LY6D expression is observed at low levels in the basal layer and high levels starting at the suprabasal layer (here referred to as epibasal), KRT4 is mid differentiation marker and CRNN stains the luminal/cell layer of terminally differentiated cell types. (F) Summary schematic of different epithelial zones of the adult esophagus with their corresponding markers identified by scRNA-seq and validated by immunofluorescence (other markers validated in FIG. 3, including LY6D higher magnification images). Scale bars represent 50 μM (Images are representative of n=3 biological replicates).

FIG. 3: Characterizing the adult human esophagus epithelium. (A) Feature plots for scRNA-seq related to FIG. 1 exhibiting molecular markers enriched in epithelial clusters: Basal cells (Cluster 2—CAV2-enriched), a suprabasal proliferative zone (Cluster 1—LY6D+), and middle-zone (Cluster 0—KRT4+). Note that TP63 is expressed throughout Clusters 1, 2, and 3 and is not specific to the basal cells. (C) Validation using immunofluorescence (IF) to identify the different zones of the human adult esophagus. CAV1 and CAV2 mark basal stem cells of the esophagus. The LY6D high expression zone starts just one cell layer above the basal cells, denoting the early differentiation-suprabasal layer. KRT4 is expressed in the mid-differentiated layer, and CRNN is expressed in the terminally differentiated luminal zone of the esophagus (see FIG. 2 for reference). ECAD is used to identify the epithelial cells, DAPI for nuclei, and TP63 is expressed broadly within the basal-early-mid zones of the esophagus. (D-E) Immunofluorescence of COL17A1 in fetal human and adult mouse esophagus. Across all tissues, COL17A1 expression was significantly restricted to the basal layer of the esophageal epithelium, where the stem-progenitor cells of the esophagus reside. Scale bars in 100 (top), 50 (middle), and 30 (lower) μm, respectively (staining for each marker combination was validated in n=3 biological replicates).

FIG. 4: Characterizing human esophageal biopsies grown in 2D in vitro at a single-cell level. (A) H&E of a representative biopsy of squamous epithelial cells of the esophagus. (B) Bright-field (BF) images of expansion of esophagus cell clusters/colonies for Day 3, Day 9, and Day 12. Scale bar represents 200 μm. (C) Proliferation as measured using the WST assay at 24, 48, 72, and 96 hrs where esophageal cells proliferate over time compared to sub-lethally irradiated 3T3-J2 mouse fibroblast cells (t-test P=0.027) (D) Doubling life of esophageal 2D in vitro cells (n=4 patients (pt)). We quantified cell numbers using two cell counting approaches, by cell counter and by DAPI counts and calculated doubling time as previously described. The average daily doubling time is 3.19 and 2.87, respectively (Cell Counts vs DAPI⁺ P=ns) (E). A total of 10550 cells grown in vitro and 4269 genes/cell were analyzed using Louvain clustering and visualized via UMAP to predict five clusters. C1/C2 express basal cell markers (See FIG. 4I), C0/3 express markers of early suprabasal cells, and C4 expresses proliferation markers. (Table S3) (F) Distribution plot of the average (x) number of cells contributing to each cluster per sample (n=3 biological replicates). (G) CDH1 expression across clusters, with (H) validation of ECAD (protein) expression suggesting an enrichment of epithelial cell types using these methods. (I) Quantification of the % of total cells that are ECAD+ in vitro in multiple patient-derived cell lines (each number on the x-axis represents a unique patient sample (n=7 pt) (top panel). Epithelial cells do not significantly increase or decrease over passage number (bottom panel) (n=3 t-test, P=not significant). (J) Feature plots of genes identified in primary tissue differential epithelial zones for basal (CAV1/COL17A1), proliferative (KI67), epibasal (LY6D), suprabasal (KRT4), and luminal cells (CRNN).

FIG. 5: In vitro cells are analogous to their in vivo counterparts and are enriched for basal cells. sc-RNA seq data from in vitro cultures derived from the match normal esophageal tissue of patients with a history of Barrett's esophagus and/or GERD. (A) Dot plots of the top 5 genes expressed in each cluster of cells in vitro. (B) Louvain clustering and UMAP visualization of predicts cell clustering for in vitro grown samples [patients (Pt #) (n=3)]. (C) Feature plots of genes associated with basal stem cells, including KRT15, TP63, CAV2, and ITGB4. (D) Clustering the individual in vitro patient samples with feature plots for CDH1 and VIM expression levels and percentage of cells expressing each marker. (E) Feature plots for scRNA-seq data for individual patient cell lines, for expression of basal cell and epithelial markers (COL17A1, KRT14, TP63, KRT15) of the esophagus per patient in vitro sample (F) Protein expression patterns for COL17A1, KRT14, TP63, KRT15 in the adult human esophagus (images courtesy of the Human Protein Atlas (Uhlen 2005; Uhlen et al. 2015) depicting expression patterns for markers stained by IHC.

FIG. 6: Expansion of human embryonic esophageal cells in vitro (A) Schematic of human fetal tissue dissociation for generation of 2D in vitro esophageal cell cultures. The stomach and esophagus are dissected, and the esophagus is mechanically and/or enzymatically digested and co-culture in HYE3NAC. We observed expansion of both epithelial colonies with surrounding mesenchyme upon bright-field images. (B) Immunofluorescent staining of representative primary human embryonic esophageal tissue (post-conception days (PCD) (top panel) with representative images of epithelial colonies expanded in the 2D format in vitro from a PCD-54 fetal sample. Scale bars represent 100 μm.

FIG. 7: Thawing, rescuing, and passaging esophageal cells in 2D in-vitro (A) Schematic summary of HYE3NAC protocol for 2D expansion and cryopreservation (B) summary of human esophageal lines in the biobank used for (C) rescue and expansion in 2D format. (D) Bright-field images of expanded cells depicting esophageal cell expansion for n=3 (adult lines from biopsies) and n=1 fetal Scale bars represent 500 μm (BF)

FIG. 8: In vitro and in vivo esophagus share a high degree of molecular similarity. (A) Louvain clustering and UMAP visualization of in vivo samples (blue-dotted line highlighting the epithelial (CDH1+) cluster vs. yellow-dotted line highlighting VIM+ cells. (B) Feature plots of genes expressed in different cells within the esophagus, including CAV1, COL17A1 KI67, LY6D, KRT4, and CRNN. (C) Distribution of cells from each human sample to each cluster. (D) The Scanpy function Ingest was used to project 2D in vitro grown cells onto the in vivo cell embedding. In vitro cells map to 5 clusters, with most cells mapping to in vivo basal (Cluster 2) and suprabasal clusters (Cluster 0). (E) Feature plots showing expression of basal cell genes (CAV1, COL17A1), proliferation-associated genes (KI67), suprabasal marker genes (LY6D), and differentiated marker genes (KRT4, CRNN). (F) Quantification of the proportion of cells from the in vivo sample in each cluster and the proportion of in vitro cultured cells that map to each in vivo cluster, demonstrating that in vitro cultured cells maintain similar basal cell proportions (Cluster 2, green) but have a larger proportion of suprabasal-like cells (Cluster 0, orange). (F) IF validation of COL17A1, CAV2, and KI67 expression co-expressed with TP63 and ECAD. Epibasal/suprabasal marker LY6D could not be detected. Scale bars represent 2 mm for COL17A1, CAV2, and LY6D and 50 μm for KI67.

FIG. 9: 2D in vitro grown esophageal cells molecularly resemble basal and epibasal cell types from the native tissue (A) Heat-map represents a direct comparison of the top 200 genes in each cluster for in vivo and in vitro esophageal cells (legend reflects cluster # and color reference from FIG. 2 & FIG. 4). The % of genes overlapping between lists is plotted, and grey boxes have zero overlaps. (B) Heat map showing the top genes (FC>2, P<0.01) for in vivo basal cells (FIG. 2A, Cluster 2—C2) and for in vitro basal cells (FIG. 4D—Cluster 1 (C1) and Cluster 2 (C2)). DST, CAV1, COL17A1, and ITGB4 are represented in both groups. (C) All epithelial cells were extracted from in vivo (day 0) cells and from cultured in vitro cells and were batch corrected using BBKNN. (D) Louvain clustering and UMAP visualization revealed four predicted clusters. Molecular characterization (See panels C, D) identifies Cluster 1 as expressing genes at the basal zone, Cluster 3 expressing proliferative genes, Cluster 0 expressing genes of the suprabasal cells, and Cluster 2 expressing genes of luminal cells. (E-F) Top 5 enriched genes expressed in each cluster with feature plots (F) for selected cluster-associated genes, including CAV1, KI67, LY6D, KRT4, and CRNN. (G) Quantification of the number of cells contributing to each cluster from in vivo or in vitro samples. There was significant enrichment for the proportion of basal cells in Cluster 1 from in vitro cells compared to in vivo cells (4.52% to 37.35, respectively). n=2 in vivo and n=3 in vitro biological replicates.

FIG. 10: Esophageal stratification in vitro using air-liquid interphase and cell density (A) Average TEER measurements for n=4 patient 2D in vitro derived cultures that were seeded onto transwell, and trans-epithelial resistance was measured over time (Ω·cm²) (B) Histologic and (C) immunofluorescent characterization of day 14 ALI cultures (D) qPCR for mRNA markers of proliferation (KI67), early and terminally differentiated (KRT4, CRNN respectively), and basal-suprabasal (TP63) between low vs. high-seeded wells from four independent lines generated (E) Immunofluorescent quantification of protein expression of early differentiation marker KRT4 in low vs. high cell density assay. (F) Representative image of immunofluorescence for TP63 and KRT4 in primary tissue biopsy of the adult human esophagus (G) Representative image of immunofluorescence for TP63 and KRT4 patient-derived in vitro esophagus primary 2D cell cultures, at low vs. high-density (top panel 2D) (bottom panel-confocal z-stack maximum projections) (H) Quantifications of cell types based on protein expression of n=3 independent patient primary tissue (from Fig. A) vs. n=3 in vitro cells at low vs. high density. % Cell types were counted by cells being positive or negative for the respective markers (x-axis) (I) Representative image of immunofluorescence for KI67 and TP53 in primary tissue biopsy of the adult human esophagus (J) Representative image of immunofluorescence at low density, human nuclei are identified by human-specific nuclear antigen (Hu-Nu; red), and co-stained with TP63 (green) are highly proliferative, marked by KI67 when compared to the same cell line plated at higher density (bottom row). Scale bars 100 μm. (K) Quantification of KI67+/TP63+ cells in low and high confluence. Less confluent cell colonies are highly proliferative compared to high density, confluent monolayers (n=3; t-test; P<0.001). All experiments were performed using at least n=3 biological replicates. Either t-test or multiple comparisons one-way ANOVA with Bonferroni Correction post-hoc analysis were used to compare the mean of groups. For in vitro cultures, normalization and percentages were calculated using double-positive cells for DAPI/Hu-Nu, to determine human cell percentages in vitro and exclude mouse feeder cells. Unpaired t-test was used to determine statistical significance (ns P<0.05, *P≤0.05, **P≤0.01, ***P≤0.001, ****P≤0.0001).

FIG. 11: High-density (3D) in vitro recapitulates basal and luminal zones of the human esophagus (A) Schematic summary of protocol for fixing and staining esophageal cells in either 2D or 3D format (B) High-density IF images of the base zone of the coverside where COL17A1 and CAV2 are co-expressed with TP63 and depicting a basal-zone in the 3D structures (C) Base and top views with a maximum projection (MIP) orthogonal view showing localization of COL17A1 at the base and CRNN at the top (D) Histogel was added to the 3D cells before fixing to prevent the loss of luminal cells during the IF protocol, images of bottom the side of histogel that was in contact with the top layer of the 3D culture shows CRNN and DAPI positive cells. (Scale bars 50 and 100 μm)

FIG. 12: Generation of 2D and 3D adult human esophagus organoids (A) Schematic of sample processing for generation of 2D or 3D adult human organoids. Patient biopsies are collected fresh on cold HYENAC medium, either alone or in combination; we mechanically or enzymatically dissociate the cells, and cells are then added to a six-well plate pre-coated with 3T3-J2i (4-24 hrs before adding human cells). 2 mL media is used to enhance 2D expansion. Low media facilities the interaction of epithelial with feeder cells and attachment for 2D expansion. High volume media facilitates the cells and aggregates to float. (B) Bright-field images of 2D colonies (top) vs. 3D floating (bottom) organoids (scale bars represent 100 μm) (C) Immunofluorescence (IF) for the epithelial marker (ECAD), basal-epibasal marker (TP63), and early differentiation marker (KRT4) in 2D (top panel) (scale bars represent 100 μm) and 2D (bottom panel) (scale bars represent 50 μm) (D) 100 μm bright field (BF) images were taken from AA or EA derived 3D organoids and area of 3D-organoids measured using FIJI. (E) Comparison of 3D-organoids size formation generated from primary biopsies vs. 3D-organoids generated after establishing a 2D culture, followed by switching to high-volume media (scale bars represent 100 μm) (F) IF volumetric z-stack confocal image of basal-epibasal marker (TP63) and early-differentiation markers (KRT4) in p3 organoids generated from 2D culture.

FIG. 13: 2D basal progenitor-stem cells form entire esophageal epithelial sphere 3D organoids in vitro (A) Schematic summary of protocol for the generation of basal-progenitor cells in 2D format, cryopreservation, and rescue for 2D/3D expansion. (B) Cell number quantifications of 3 patient (pt) lines thawed and rescued in either 2D, 3D (suspension vs. Matrigel)(cells were counted when 2D wells reached 90-100% confluency and needed to get passage to avoid differentiation (time points vary by doubling rates per patient). (C) 2D expanded cells were passaged to either 3D (suspension vs. Matrigel) or replated in 2D format (3T3-J2i). (D) By day 6, growth was observed in all conditions by bright-field (BF) images (E) Quantification of the area of the sphere of 3D organoids of suspension vs. Matrigel over time (F) Day 6, whole mounts IF stains were performed on suspension vs. Matrigel and maximum projection confocal images for CRNN (luminal)(arrows), COL17A1 (basal), KI67 (proliferation), and ECAD (epithelial) are shown (G) Quantitation of % KI67⁺ cells in suspension vs. Matrigel (H) Area of 3D spheres over the span of 25 days was measured and quantified for sphere vs. Matrigel (I) BF images and H&E stains on Day 25 where Matrigel spheres were collected and analyses for (J) IF of markers COL17A1 (basal), LY6D (epibasal-early), KRT4 (mid) and CRNN (late-luminal differentiation) depicting the complete epithelial formation of esophageal organoids. Scale bars in BF images are 500 μm and IF ranges from 100 to 50 μm.

FIG. 14: Mapping the adult human esophagus in vivo and in vitro. (A) Schematic representation describing and summarizing the findings of this study.

FIG. 15: Clinical Characteristics of Primary Human Esophageal Samples for 2D or 3D in vitro culture.

### DETAILED DESCRIPTION OF THE INVENTION

Many esophageal diseases can arise during development or throughout life, caused by injury and inflammation. Therefore, well-characterized in vitro models and detailed methods are essential for studying human esophageal development, homeostasis, and disease. Experiments conducted during the course of developing embodiments for the present invention aimed to 1) create an atlas of the cell types observed in the normal adult human esophagus; 2) establish an ancestrally diverse biobank of in vitro esophagus tissue to interrogate homeostasis and injury, and 3) to benchmark in vitro models using the adult human esophagus atlas. To these ends, a single-cell RNA sequencing (scRNA-seq) reference atlas was created using fresh adult esophagus biopsies and a continuously expanding biobank of patient-derived in vitro cultures (n=55 lines). Several transcriptionally distinct cell classes in the native human adult esophagus were identified and validated, with four populations belonging to the epithelial layer, including basal, epibasal, early differentiating, and terminally differentiated-luminal cells. Benchmarking in vitro esophagus cultures to the in vivo reference using scRNA-seq showed that the basal stem cells were robustly maintained in vitro, and the diversity of epithelial cell types in culture is dependent on cell density. Low-density cultures possess an abundance of basal cells in a highly proliferative state, whereas increased density leads to more luminal and differentiated cells. Such experiments further demonstrated that cultures can be grown in 2D or as 3D organoids, and these methods can be employed for modeling the complete epithelial layers, thereby enabling in vitro modeling of the human adult esophagus.

Accordingly, the invention disclosed herein generally relates to methods and systems for culturing and expanding esophageal cells with a growth media comprising a rho-kinase inhibitor, a WNT-activator/agonist, EGF, an inhibitor of TGFβ signaling, an inhibitor of BMP signaling, and hydrocortisone.

In certain embodiments, the present invention provides methods comprising culturing in vitro obtained esophageal cells with a growth media comprising a rho-kinase inhibitor, a WNT-activator/agonist, EGF, an inhibitor of TGFβ signaling, an inhibitor of BMP signaling, and hydrocortisone, wherein the culturing results in expansion of the obtained esophageal cells.

In certain embodiments, the present invention provides methods of generating stratified esophageal epithelium, comprising culturing obtained esophageal cells in vitro, wherein the culturing results in expansion of the obtained esophageal cells into stratified esophageal epithelium, wherein the culturing comprises a growth medium comprising hydrocortisone, epidermal growth factor (EGF), a rho-kinase inhibiting agent, a Wnt signaling pathway activating agent, a TGFβ inhibiting agent, and a BMP inhibiting agent.

Such methods are not limited to a particular Wnt signaling pathway activating agent within the growth medium. In some embodiments, the Wnt signaling pathway activating agent is a small molecule or agonist that activates the Wnt signaling pathway. In some embodiments, the small molecule or agonist that activates the Wnt signaling pathway is CHIR99021. In some embodiments, activating the Wnt signaling pathway occurs through culturing the esophageal cells with one or more molecules configured to activate a Wnt protein, wherein the Wnt protein is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, activating the Wnt signaling pathway comprises culturing the esophageal cells with a small molecule or other agonist that stimulates Wnt signaling. In some embodiments, the Wnt agonist is CHIR99021.

Such methods are not limited to a particular rho-kinase inhibiting agent within the growth medium. In some embodiments, the agent is a small molecule or antagonist that inhibits rho-kinase. In some embodiments, the rho-kinase inhibiting agent is Y-276327.

Such methods are not limited to a particular BMP inhibiting agent within the growth medium. In some embodiments, the BMP inhibiting agent is a small molecule or antagonist that inhibits the BMP signaling pathway. In some embodiments, the small molecule or antagonist that inhibits the BMP signaling pathway is Noggin.

Such methods are not limited to a particular TGFβ inhibiting agent within the growth medium. In some embodiments, the TGFβ inhibiting agent is a small molecule or antagonist that inhibits the TGFβ signaling pathway. In some embodiments, the small molecule or antagonist that inhibits the TGFβ signaling pathway is A-8301. In some embodiments, the small molecule or antagonist that inhibits the TGFβ signaling pathway is SB431542.

In some embodiments, the growth medium comprises hydrocortisone, EGF, Y-276327, CHIR99021, A-8301, and noggin.

In some embodiments, the expanded esophageal cells possess esophageal stem cells and differentiated esophageal cells. In some embodiments, the expanded esophageal cells are capable of cryopreservation. In some embodiments, the cryopreserved expanded esophageal cells can be thawed for purposes of generation of esophageal cell lines (e.g., 2-dimensional). In some embodiments, the cryopreserved expanded esophageal cells can be thawed for purposes of generation of esophageal organoid tissue (e.g., 3-dimensional).

In some embodiments, the expanded esophageal cells are stratified esophageal epithelium cells. In some embodiments, the stratified esophageal epithelium is 2-dimensional stratified esophageal epithelium. In some embodiments, the stratified esophageal epithelium is 3-dimensional stratified esophageal epithelium (e.g., 3-dimensional esophageal organoid tissue).

In some embodiments, the culturing further comprises seeding the obtained esophageal cells onto sub-lethally irradiated feeder cells and the growth media. In some embodiments, the sub-lethally irradiated feeder cells are 3T3-J2i sub-lethally irradiated feeder cells.

In some embodiments, the culturing further comprises seeding the obtained esophageal cells onto solubilized basement membrane matrix cells and the growth media. In some embodiments, the solubilized basement membrane matrix cells are matrigel cells.

Such embodiments are not limited to utilizing a specific amount of growth medium within the culturing step.

In some embodiments, the amount of growth medium utilized in the culturing step is approximately 2 ml within a 9.8 cm² well, which results in generation of 2-dimensional stratified esophageal epithelium.

In some embodiments, the amount of growth medium utilized in the culturing step is approximately 4 ml within a 9.8 cm² well, which results in generation of 3-dimensional stratified esophageal epithelium (e.g., 3-dimensional esophageal organoid tissue).

In some embodiments, the culturing of obtained esophageal tissue with the growth medium occurs over a specified temporal period. In some embodiments, the culturing of obtained esophageal tissue with the growth medium occurs for 6 or more hours; 12 or more hours; 18 or more hours; 24 or more hours; 36 or more hours; 48 or more hours; 60 or more hours; 72 or more hours; 84 or more hours; 96 or more hours; 120 or more hours; 150 or more hours; 180 or more hours; or 240 or more hours.

In some embodiments, the obtained esophageal cells are cultured with the growth medium at a concentration of ng/ml or higher, 20 ng/ml or higher; 50 ng/ml or higher; 75 ng/ml or higher; 100 ng/ml or higher; 120 ng/ml or higher; 150 ng/ml or higher, 200 ng/ml or higher; 500 ng/ml or higher; 1,000 ng/ml or higher; 1,200 ng/ml or higher, 1,500 ng/ml or higher; 2,000 ng/ml or higher, 5,000 ng/ml or higher, 7,000 ng/ml or higher, 10,000 ng/ml or higher; or 15,000 ng/ml or higher. In some embodiments, concentration of the growth medium is maintained at a constant level throughout the culturing. In other embodiments, concentration of the growth medium is varied during the course of the culturing.

Such embodiments are not limited to a particular type or kind of obtained esophageal cells. In some embodiments, the obtained esophageal cells are comprised within a biopsy sample from a subject (e.g., a human subject). In some embodiments, the obtained esophageal cells are comprised within a surgical specimen sample from a subject (e.g., a human subject). In some embodiments, the obtained esophageal cells are fetal esophageal cells. In some embodiments, the obtained esophageal cells are adult esophageal cells. In some embodiments, the obtained esophageal cells are esophageal stem cells. In some embodiments, the obtained esophageal cells are differentiated esophageal cells.

In some embodiments, prior to the culturing step the obtained esophageal cells are minced or enzymatically digested.

In certain embodiments, the present invention provides compositions comprising or consisting of esophageal cells expanded with the methods described herein.

In certain embodiments, the present invention provides kits comprising or consisting of stratified esophageal epithelium, 2-dimensional stratified esophageal epithelium, and/or 3-dimensional stratified esophageal epithelium (e.g., 3-dimensional esophageal organoid tissue) produced in vitro from the described methods.

In some embodiments, the expanded esophageal cells produced in vitro from the described methods can be used to screen drugs for esophageal tissue uptake and mechanisms of transport. For example, this can be done in a high throughput manner to screen for the most readily absorbed drugs, and can augment Phase 1 clinical trials that are done to study drug esophageal tissue uptake and esophageal tissue toxicity. This includes pericellular and intracellular transport mechanisms of small molecules, peptides, metabolites, salts.

In some embodiments, the expanded esophageal cells produced in vitro from the described methods can be used to identify the molecular basis of normal human esophageal development.

In some embodiments, the expanded esophageal cells produced in vitro from the described methods can be used to identify the molecular basis of congenital defects affecting human esophageal development.

In some embodiments, the expanded esophageal cells produced in vitro from the described methods can be used to correct esophageal related congenital defects caused by genetic mutations. In particular, mutations affecting human esophageal development can be corrected using the expanded esophageal cells produced in vitro from the described methods. In some embodiments, the expanded esophageal cells produced in vitro from the described methods can be used to generate replacement tissue.

In some embodiments, the expanded esophageal cells produced in vitro from the described methods can be used to generate replacement esophageal tissue for esophageal related disorders.

In some embodiments, a diagnostic kit or package is developed to include the expanded esophageal cells produced in vitro from the described methods and based on one or more of the aforementioned utilities.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. The use of pronouns such as “I”, “we”, and “our”, for example, refer to the inventors.

### Example I

This example demonstrates that adult human esophagus epithelium contains molecularly distinct zones defined by scRNA-seq and validated at the protein level.

The esophagus is composed of stratified squamous epithelium, underlying stromal tissue (lamina propria), and smooth muscle (muscularis mucosae) (Rosekrans et al. 2015). To characterize the esophagus, we obtained normal/healthy squamous epithelial (normal squamous—“NS”) adult human biopsies (approximately 3 mm²) (n=2 independent patients, with n=3-4 biopsies used for dissociation), carried out enzymatic dissociation into single cells, which were captured using the 10× Chromium platform for subsequent sequencing. Louvain clustering defined seven transcriptionally distinct clusters (from 0 to 6) present in the biopsies (FIG. 1A-C). Using well-established marker genes, we defined classes of cells as Epithelial (CDH1⁺—Clusters 0, 1, 2, 3) or stroma/lamina propria (VIM⁺—Clusters 4, 5, 6) which included immune cells (Clusters 4/5) (FIG. 1B-F). The contribution to each cluster was consistent across biological replicates (FIG. 1B), and classifications of each cluster were performed by cross-referencing the Human Protein Atlas (FIG. 1G) (Uhlen 2005).

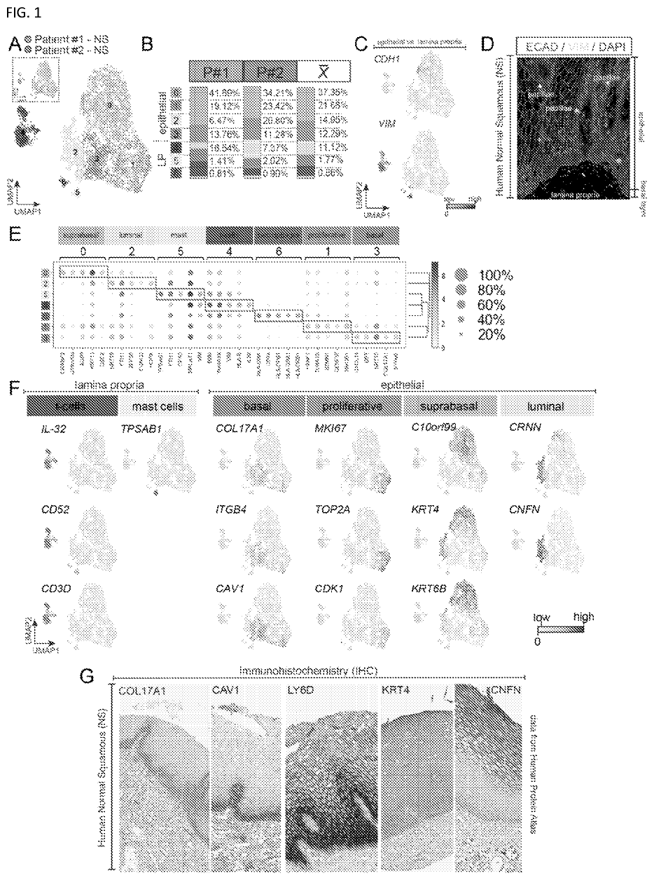

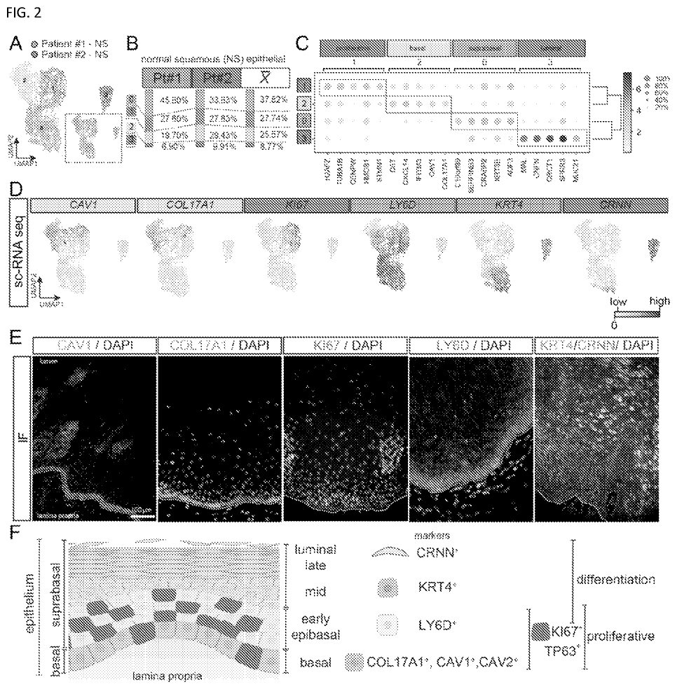

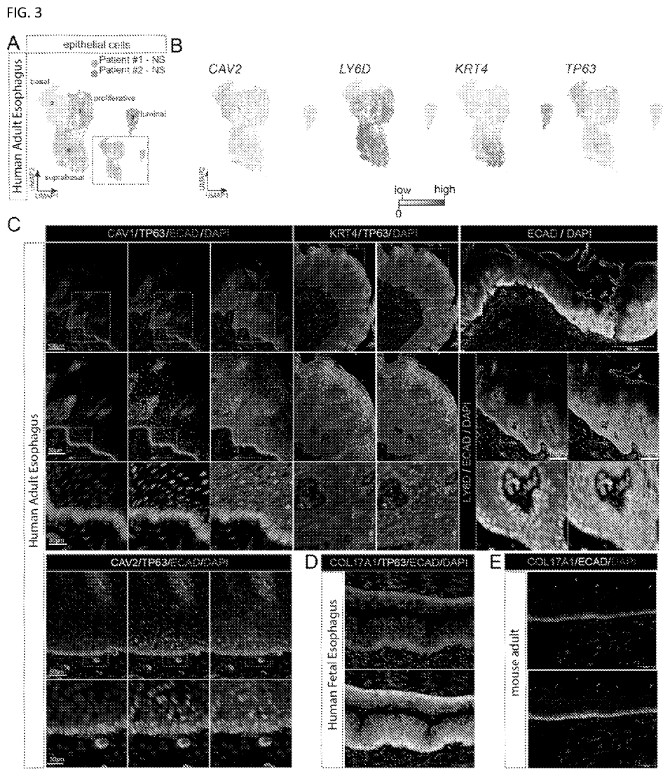

To further characterize the epithelium, epithelial clusters (0, 1, 2, 3) were computationally extracted and re-clustered, revealing four predicted sub-clusters (FIG. 2A). Individual biopsies contributed to each cluster in similar proportions (FIG. 2B). Unsupervised clustering was used to plot the top 5 genes in an unbiased way (FIG. 2C) and used to determine that epithelial clusters correspond to basal cells (Cluster 2), proliferative (Cluster 1), suprabasal (Cluster 0), and the differentiated/luminal zone (Cluster 3), (FIG. 2C, FIG. 3A). We observed that TP63, a marker canonically used to identify basal cells, was broadly expressed in basal, proliferative, and suprabasal cell clusters (FIG. 3B). Protein staining validated this finding, which showed broad epithelial expression (FIG. 3C). scRNA-seq data identified genes that were highly enriched in the basal cell cluster (Cluster 2) (FIG. 2C-D), which had specific high expression and localization to the basal cell layer by immunofluorescence, and included newly identified markers CAV1 and CAV2, alongside COL17A1 which has recently been identified as an esophagus stem cell marker (Bogte et al. 2021) (FIG. 2C-E, FIG. 3). We additionally observe high COL17A1 expression in the adult mouse esophagus and human fetal basal cell zone of the esophagus (FIG. 3D-E)). scRNA-seq identified LY6D as an enriched marker in the suprabasal/early squamous/differentiated cells and proliferative cluster (Clusters 0, 1) (FIG. 2D, FIG. 3). Supporting this, immunofluorescence shows that LY6D protein is most highly expressed in the cell layers just above the basal cell domain, which has been referred to as the epibasal/early differentiation domain (Zhang et al. 2021) (FIG. 2E, FIG. 3C), and which is also where the majority of KI67⁺ proliferative cells are observed (FIG. 2E). Within the suprabasal cluster (Cluster 0), we observed that KRT4 is expressed in a low-to-high gradient (FIG. 2D). At the protein level, KRT4 marks the mid-suprabasal epithelium above the LY6D⁺ epithelium and below CRNN^{HI} differentiated luminal cells (FIG. 2E, FIG. 3C). Finally, Cluster 3 represents a CRNN^{HI} zone that marks terminally differentiated luminal cells in the portion of the squamous epithelium near the lumen (FIG. 2D-E). Our data is consistent with recent publications characterizing human esophageal adult tissue using the single-cell transcriptomics (Bogte et al. 2021; Madissoon et al. 2019), and additionally provides identification of new basal cell markers (i.e., CAV1, CAV2), an epibasal marker (LY6D), and detailed cross-validation of mRNA signatures with protein markers to map different zones of the esophagus (FIG. 2F).

### Example II

This example demonstrates rapid expansion and long-term maintenance of patient-derived esophageal basal cells in 2D cultures.

Over the past several years, work has revealed that long-term culture of human epithelial tissues in vitro requires culture conditions that support epithelial stem cell self-renewal. Using this framework, 2D and 3D models of the human esophagus have been established from iPSCs and primary tissue (Trisno et al. 2018; Bailey et al. 2019; Yamamoto et al. 2016; Kasagi et al. 2018; Liu et al. 2017). Although 3D organoids have advantages, it has also been reported that esophagus organoids are particularly slow-growing and have been reported to have a low terminal passage, with no evidence of recovery after cryopreservation (Kasagi et al. 2018). 2D culture of stratified squamous epithelia such as skin and esophagus, and other organs with basal stem cells such as lung, have also been successful (Mou et al. 2016; DeWard, Cramer, and Lagasse 2014). However, the establishment of 2D cultures and single-cell benchmarking to the native human esophagus have not been carried out. We, therefore, aimed to unify previous methods to achieve a long-term culture of the primary human esophagus and to compare in vitro grown cells to the native in vivo esophagus.

To determine optimal conditions for primary esophagus cell expansion, we screened many combinations of media plus growth factors with a primary outcome measure of robust cell survival and expansion. Our most successful condition for long-term adult human esophageal cultures utilized sub-lethally irradiated feeder cells (3T3-J2i) (Suprynowicz et al. 2017) with the rho-kinase inhibitor (Y-27632) plus CHIR99021, EGF, and dual SMAD inhibition using inhibitors of TGFb (A-8301) and BMP (Noggin). Finally, we tested the addition of hydrocortisone (H) based on previous studies culturing stratified skin epithelium in vitro (Vaughan, Kass L. L., and Uzman 1981). We refer to this culture condition as HYENAC (Hydrocortisone, Y-276327, EGF, NOG, A-8301, CHIR99021) (see methods for details).

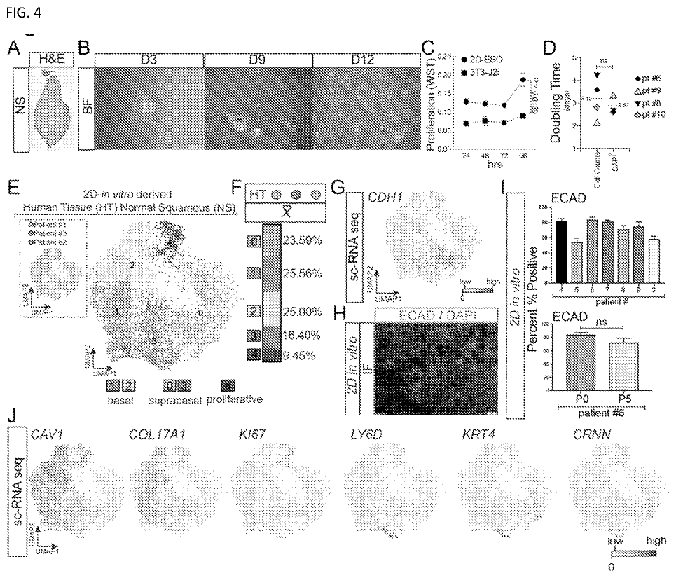

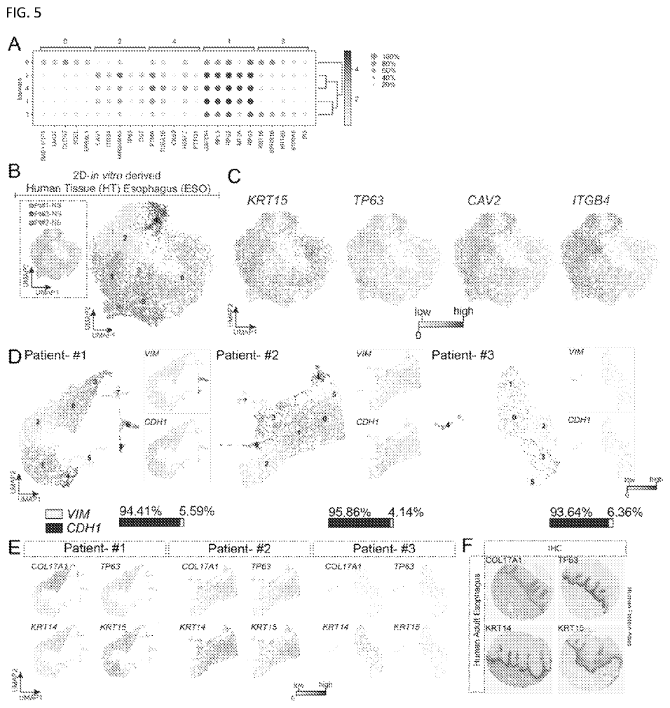

To demonstrate the ability of HYENAC media to support long-term esophagus cultures, matched cells from biopsies used to generate scRNA-seq data in FIG. 2 were plated on the day biopsies were obtained (Day 0—D0). All normal tissues used to generate 2D in vitro cell lines (Table 1) were either finely minced or enzymatically digested to produce single cells or small cell clusters before plating. Both single cells and cell clumps attached and expanded as small colonies, which eventually grew to confluence (FIG. 4A-B). Irradiated 3T3-J2i cells do not proliferate, while esophageal cells continue proliferating over time (FIG. 4C). We observed an average population doubling time in the 2D expansion system of 2.6 days (FIG. 4D). This doubling time is consistent with in vivo mouse esophagus studies showing that basal stem cells proliferate on average every 2-3 days (Piedrafita et al. 2020; Doupé et al. 2012). After three passages and 30-40 days in culture, we assessed in vitro cultures using scRNA-seq (n=3). We applied Louvain clustering to reveal five predicted clusters (FIG. 4E), and plotted the proportion of total cells within each cluster (FIG. 4F). All clusters were enriched for the epithelial marker CDH1, with little non-epithelial (VIM⁺) expansion (average of biological replicates CDH1+96.6% vs VIM+5.4% (FIG. 5E) and ECAD staining revealed a high percentage of epithelial cells (FIG. 4G-I), a finding that was quantitated via protein staining (% ECAD+ cells, n=7 independent lines, FIG. 4I top) demonstrating ˜50-80% of cells were ECAD+. The proportion of ECAD+ cells did not change across passages (FIG. 4I, bottom). The top 5 most highly enriched genes were plotted (FIG. 5A) and identified that Cluster 4 contains a proliferative signature, Clusters 1 and 2 expressed markers of basal-stem cells including CAV1, CAV2, and COL17A1 and other known markers such as TP63, KRT15, and ITGB4 (DeWard, Cramer, and Lagasse 2014; Bogte et al. 2021) (FIG. 4J, FIG. 5B-C). We observed that clusters 0 and 3 had cells that expressed low levels of markers typical of the epibasal, early, and late differentiation zones (FIG. 4J).

TABLE 1
Clinical Characteristics of Primary Human Esophageal Samples for 2D or 3D in vitro Culture

| | | | | | race | | | | | | | | | |
|---|---|---|---|---|---|---|---|---|---|---|---|---|---|---|
| | Esophagus | | | | African American [symbol] | | Asian [symbol] | | Caucasian | | Hispanic | | | |
| Procedure | Disease | n | gender | age | male | female | male | female | male | female | male | female | no | other [symbol] |
| Normal [symbol] biopsy | [symbol] | [symbol] | female [symbol] | [symbol] | [symbol] | [symbol] | [symbol] | [symbol] | [symbol] | [symbol] | [symbol] | [symbol] | data | tissue |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

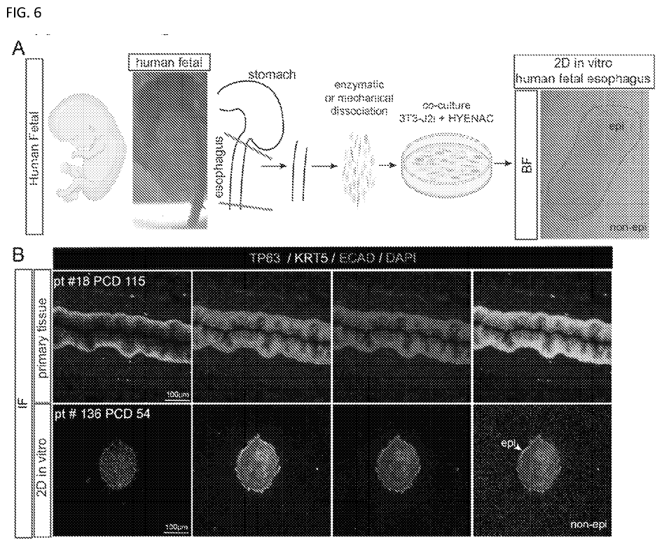

We observed that these culture conditions supported 2D in vitro culture of human fetal esophageal cells (samples used ranged from 54-100 post-conception days (PCD)). Fetal tissue was mechanically and/or enzymatically digested and cultured as described for adult tissue (FIG. 6A). We observed expansion of both epithelial and mesenchymal cells (FIG. 6A). Immunofluorescent staining for ECAD, TP63, and KRT5 revealed the expansion of epithelial basal/epibasal colonies in vitro (FIG. 6B).

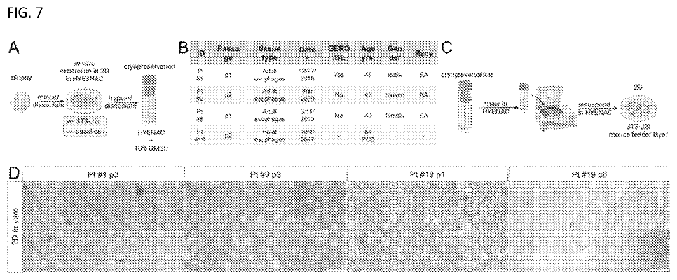

We then sought to determine if esophagus cell lines could be cryopreserved using HYENAC in combination with standard protocols for freezing (FIG. 7A). We thawed four lines that were established and were in cryopreservation for different lengths of time, and from different age groups, sexes, and races (FIG. 7B). All four samples were successfully thawed, expanded, and passaged (FIG. 7D). In addition, for fetal samples, we observed the expansion of both epithelial and mesenchyme cell types after thawing (FIG. 7D), consistent with the matched fresh sample (FIG. 6).

### Example III

This example demonstrates 2D in vitro grown esophageal cells molecularly resemble basal cells from native tissue.

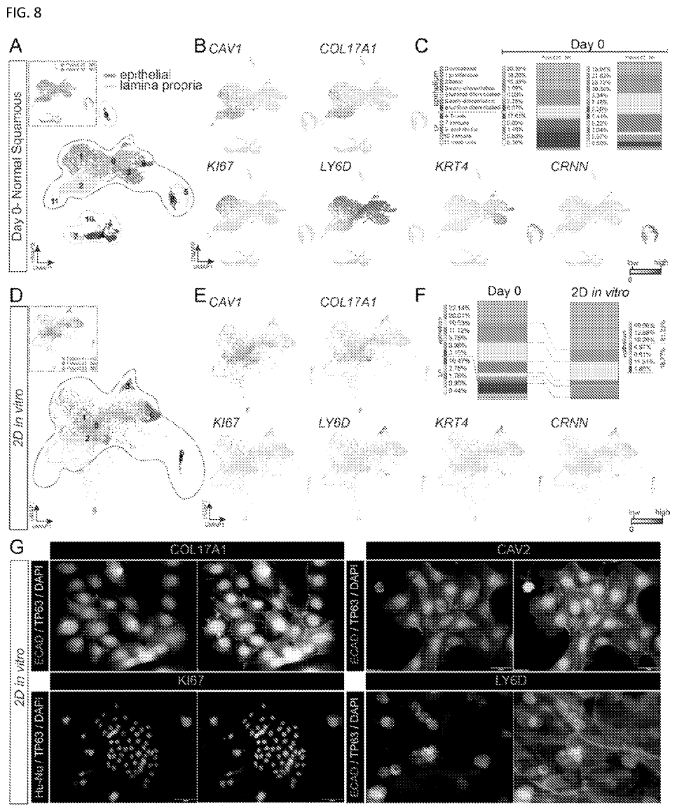

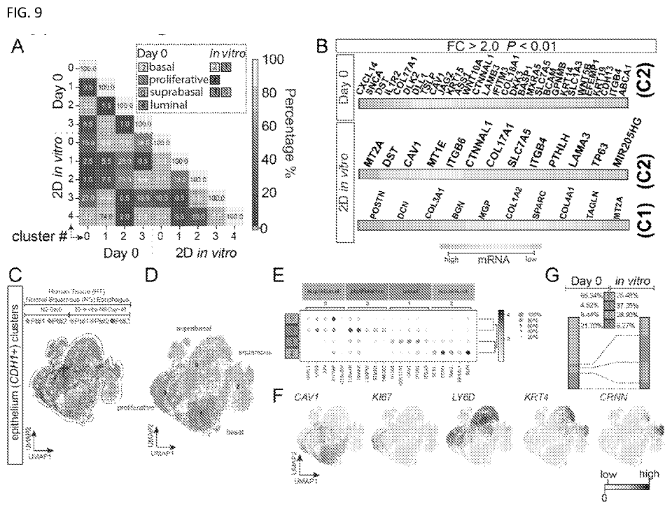

To further interrogate how closely in vitro samples resemble in vivo esophageal tissue, we carried out three complementary but separate analyses using the scRNA-seq data and validated protein levels (FIG. 8, FIG. 9). First, we directly compared the single-cell transcriptomes of in vivo samples (described in FIG. 2) with in vitro data (described in FIG. 4) (FIG. 9A-B). Second, we integrated in vitro and in vivo data following batch correction to analyze the samples in one analysis (FIG. 9C-F). Third, we performed label transfer using Ingest (Wolf, Angerer, and Theis 2017), using the in vivo UMAP embedding as a high dimensional search space and projecting in vitro samples onto the in vivo map (FIG. 8A-F). To directly compare data from in vivo Day 0 (fresh biopsies) vs in vitro cultures, we obtained gene lists of the most statistically enriched genes in each cluster (from FIG. 2 and FIG. 4) and examined gene overlap in the enrichment lists. The most highly similar clusters were proliferative (74% overlap) followed by the basal cell clusters (FIG. 9A-B). The in vivo basal cell cluster (Cluster 2) overlapped most closely with in vitro Cluster 1 and 2 with 39% and 33% overlap, respectively. The suprabasal cluster and differentiated in vivo clusters (Clusters 0 and 3) had the highest shared similarity to the differentiating in vitro clusters (Clusters 0 and 3) (FIG. 9A). With respect to the basal cell clusters (C2 in vivo, C1/C2 in vitro), overlap included many basal cell markers that were common between clusters (i.e., CAV1, CAV2, ITGB4, COL17A1) (FIG. 9B). Next, we directly compared samples by integrating and batch correcting in vivo (epithelium only) and in vitro data with BBKNN (Polanski et al. 2020), followed by clustering (FIG. 9C-G). Integrated data generated four predicted clusters (Figure S6D-E), which could be assigned to proliferative (Cluster 3), basal (Cluster 0), suprabasal (Cluster 1), and luminal (Cluster 2) cell types based on expressed genes (FIG. 9C-F). Both in vitro and in vivo cells contributed to each cluster (FIG. 9C), however, the distribution from in vitro or in vivo cells was not equal for all clusters. For example, only ˜4.5% of cells were designated as basal cells (Cluster 1) from the in vivo sample whereas ˜37.4% of cells were assigned to this cluster from the in vitro sample (FIG. 9G). Lastly, we re-clustered the in vivo data (entire data set, including stroma/immune) (FIG. 8A-C), and then used the Ingest function in ScanPy to project in vitro cells onto the in vivo UMAP embedding (FIG. 8D-F). This analysis revealed that most 2D in vitro cells mapped to the proliferative, basal, and epibasal cell clusters of the in vivo search space, with far fewer cells mapping to the differentiated mid-luminal cells (FIG. 8E-F), as highlighted by distribution plots comparing the proportion of cells from in vivo tissue to each cluster versus the projected proportion from in vitro cells (FIG. 8F). Taken together, the data suggest that 2D expanded esophageal cells closely resemble the basal-epibasal cells observed in the human adult esophagus.

To validate these observations, we performed IF on 2D cells for basal (COL17A1, CAV2), basal-epibasal (TP63), epibasal (LY6D), proliferative (KI67), and total epithelial (ECAD) markers. The 2D cells show positive co-expression of basal (COL17A1, CAV2), basal-epibasal (TP63), epithelial (ECAD) markers (FIG. 80, top), and proliferation positive (KI67⁺)(FIG. 8G, top). Interestingly, even though the mRNA single-cell data suggested two in vitro clusters (C0, C3)(FIG. 4) expressing suprabasal genes, we did not observe LY6D positive expression at the 2D state. Suggesting, that the 2D expansion method enhances proliferative basal progenitor/stem cells of the esophagus.

### Example IV

This example demonstrates Air Liquid Interface (ALI), high density culture, and 3D growth in Matrigel® drive stratification.

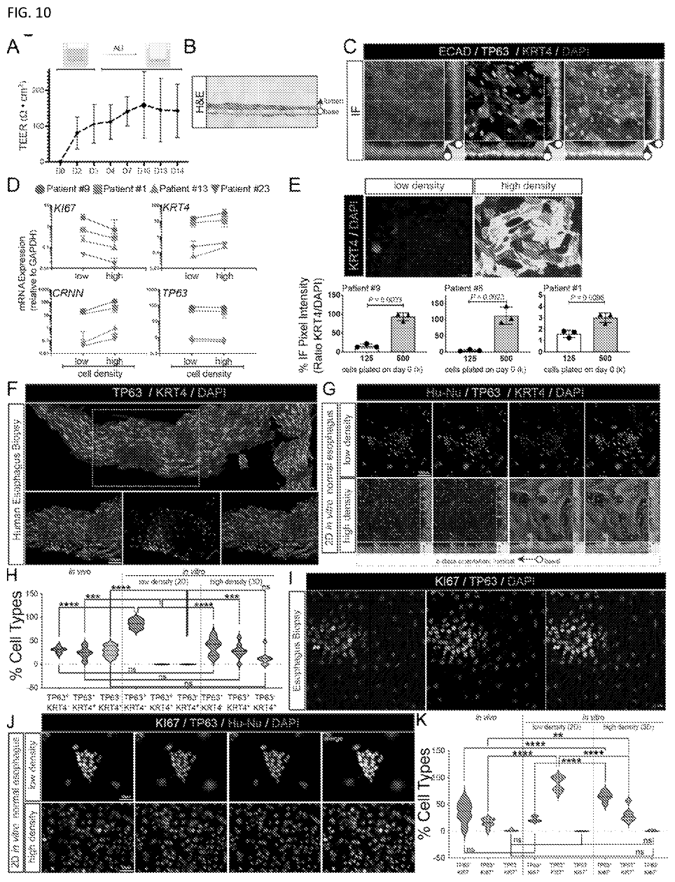

Media conditions presented above enriched basal stem cells in vitro; however, the human esophagus is comprised of stratified epithelial layers that encompass distinct zones of differentiated cells in layers as they approach the lumen. Air-liquid interface (ALI) has been used (Blevins et al. 2018), where cells are seeded in transwells, and subsequently exposed to air in the upper chamber (5% CO₂, 95% ambient), leading to the formation of a stratified epithelium (Yamamoto et al. 2016). This assay is valuable because the transwell format further allows physiological measurements of the epithelial barrier (Kleuskens et al. 2021; Blevins et al. 2018). Using transwells, we generated ALI cultures as previously described (Srinivasan, Shuler, and Hickman 2015) and measured the trans-epithelial electrical resistance (Ω·cm² (TEER), n=4)(FIG. 10A). We observed a significant increase in TEER by day 2 of seeding cells (from 0 to 80.2 (12 cm²), p=0.001), and, after removing media from the apical chamber on Day 4, we observed a steady increase of TEER from Day 2 to Day 14 (D2 80.2 vs D14 142.4 (Ω·cm²), p=0.1109 ns) (FIG. 10A). The average TEER across all days in ALI was 126 (Ω·cm²) (n=4). On day 14, transwells were fixed for histologic and immunofluorescence staining for TP63 and KRT4 (FIG. 10B-C). We observed that by day 14 transwells had formed a multilayer tissue (FIG. 10B), with a basal layer of TP63⁺ cells at the bottom of the well and KRT4+ cells on top (FIG. 10B-C).

During our experiments, we observed that when cells became overly confluent, they appeared to pile on top of each other. To investigate confluent/dense cultures in a reproducible manner, we varied the number of cells plated at Day 0 (FIG. 10D-L). Four independent cell lines (n=4) were plated on coverslips at 125,000 cells/well (low density) or 500,000 cells/well (high density) and were analyzed 5 days later (FIG. 10D). We observed that by day 5, the high-density wells had lower expression of KI67, and increased expression of differentiation/stratification markers such as KRT4 and CRNN when compared to the low-density plated wells (FIG. 10D). We did not observe a change in mRNA expression of the basal/epibasal marker TP63 (FIG. 10D). We fixed the cells and confirmed the expression of KRT4 by quantifying the pixel intensity of the protein in the high-density wells, and in the low-density wells (where expression was absent) (FIG. 10E). These data suggest that cell density and confluency of cells lead to differentiation and stratification of cells, without the need for ALI in transwells.

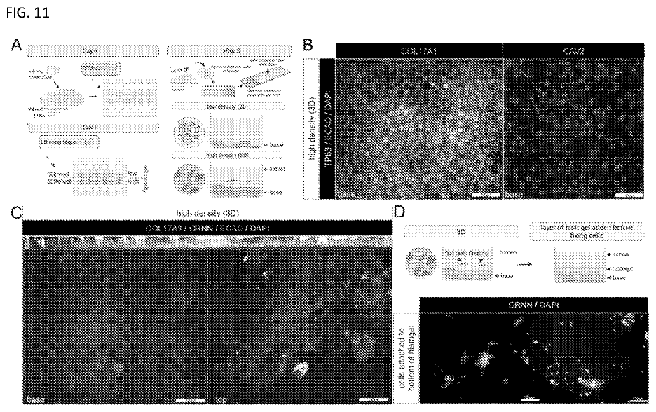

Given that TP63 and KRT4 are broad markers, with TP63 marking basal, and epibasal cells, while KRT4 marks mid and late differentiating cells, we used this combination of markers to delineate basal/epibasal cells (TP63⁺ KRT4⁻), mid differentiating cells (TP63⁺ KRT4⁺) and late differentiating cells (TP63⁻ KRT4⁺). When we examined tissue biopsies, we observed that all populations of cells were distributed similarly (p>0.05, ns) (FIG. 10G-I). Meanwhile, in the low-density in vitro cell population, we see a statistically significant enrichment in TP63⁺ KRT4⁻ cells when compared to the in vivo tissue (86.04% low-density in vitro vs. 30.99% in vivo; p<0.0001), with little presence of the other two populations (FIG. 10G-I). In the high-density culture, we observe a cellular distribution similar to that of the in vivo tissue: the TP63⁺ KRT4⁻ cells have an average of 38.4% in the high-density in vitro vs. 30.99% in the in vivo, the TP63⁺ KRT4⁺ double-positive cells have an average of 28.43% in the high-density in vitro vs. 24.66% in the in vivo, and the differentiated TP63⁻ KRT4⁺ cells have an average of 38.12% in the high-density in vitro vs. 13.45% in vivo (FIG. 10G-L). Altogether, high-density culture conditions lead to proportions of basal, early, and late differentiating cells similar to in vivo tissue (FIG. 10I), without the need for ALI. We similarly quantified the proportion of TP63+ and TP63− cells that co-expressed KI67 (TP63⁺ KI67⁺; TP63⁺ KI67⁻; TP63⁻ KI67⁺) in culture and in vivo. We observed that the biopsy tissue contained TP63⁺ cells that were non-proliferative (TP63⁺ K167⁻ 38.42%), which was higher than the population of TP63⁺ KI67⁺ proliferative cells (16.83%), and proliferative TP63⁻ cells were rarely observed (TP63⁻ KI67⁺, 1.80%) (FIG. 10J-L). In low-density cultures, we observed significant enrichment of the basal-proliferative TP63⁺ KI67⁺ population whereas the distribution of high-density cultured cells more closely reflected the in vivo distributions (FIG. 10L). Altogether, this suggests that cells maintained at low confluency are enriched for proliferative TP63⁺ cells whereas cells at high confluency reflect the range of basal/epibasal and differentiated cells found in the stratified adult human tissue. Nonetheless, we had described that TP63⁺ and KRT4⁺ are broad markers of basal-epibasal and suprabasal-luminal of the adult esophagus (FIG. 2). Therefore, we investigated if the high confluency in vitro protocol led to expression of COL17A1 (basal) vs CRNN (luminal) markers (FIG. 11). Expression of COL17A1 was restricted to the base of the high-density culture while CRNN expressing cells were sparse and observed at the top of the stratified layers (FIG. 11A). Of note, we observed that while fixing and staining the high-density cultures, there were populations of cells that were floating in the media and washed off from the coverslips. Therefore, to test if the process was causing loss of the CRNN⁺ population, we added a thin layer of histogel to the top of the coverslip before fixing cells. After IF staining, we peeled off the histogel and observed flat-shaped CRNN cells attached to the histogel (FIG. 11D). Suggesting that in the process of staining and fixing cells, the luminal cells are detaching and being sloughed off into the media, a phenomenon that is observed normally in the human esophagus.

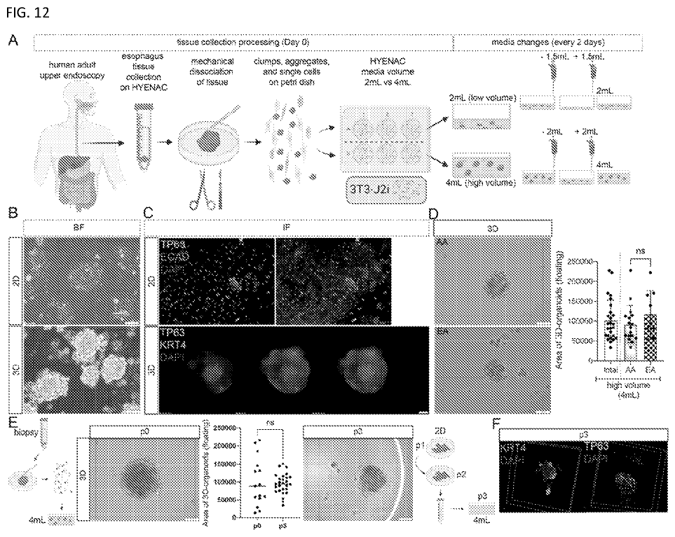

During the process of generating the esophageal 2D lines, we observed that while passaging cells, increasing media volume led to floating structures that resembled a 3D-sphere (FIG. 12). After plating dissociated biopsies onto 6-well plates (9.8 cm²), wells were either given a low volume of media (2 mL) or a high-volume media (4 mL), and the media was changed every two days (FIG. 12A). We observed that low media volume led cells to attach to the tissue culture plate whereas high media volume led to the formation of organoids (FIG. 12B). Immunofluorescence of organoids after 30 days revealed an ‘inside out’ structure with the basal/epibasal marker TP63 localized on the inside of the organoid while the differentiation marker KRT4 was localized on the outer side towards the media (FIG. 12C). This polarity has been demonstrated with intestinal organoids in suspension culture in the absence of matrix cues (Co et al. 2019; Capeling et al. 2022). This phenomenon was observed in multiple patient lines (FIG. 12D), and we found that the 3D-floating organoids could be passaged (FIG. 12E). Lastly established 2D cultures could be seeded into low-attachment plates and gave rise to organoids with a similar organization (FIG. 12E), and we observed no difference in size localization co cells within organoids generated from 2D compared to those generated from primary tissue (FIG. 12E-F). These data show that 3D floating organoids can be generated from primary tissue, or from 2D cultured cells, and resemble the correct polarity observed in the human esophagus.

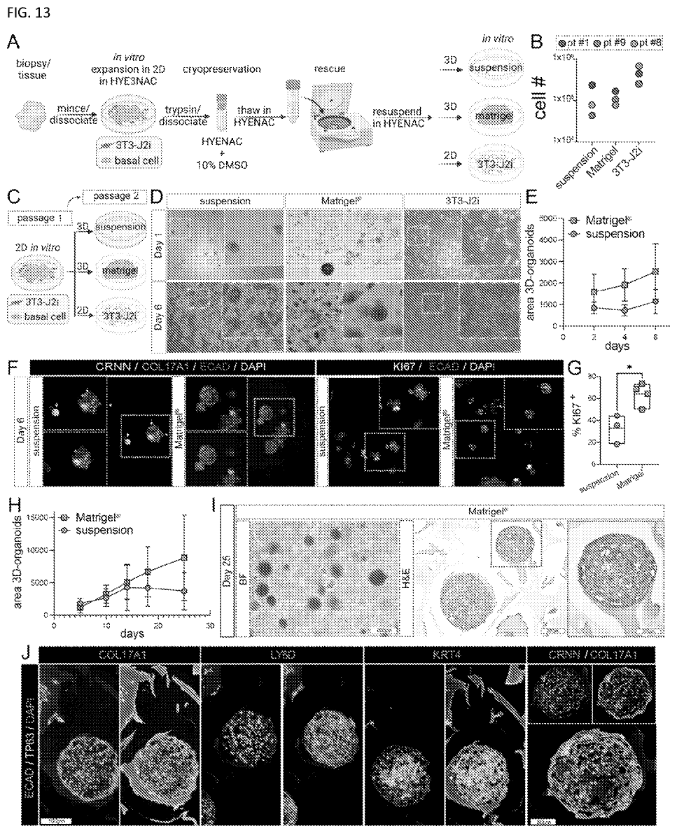

Finally, we sought to interrogate the potential of cryopreserved 2D cells to recover and form 3D organoids (FIG. 13A). We thawed cells and cultured them in either Matrigel, on low-attachment plates (referred to as suspension culture) or plated them into 2D culture on 3T3-J2i feeder cells (n=3 patient lines). We counted cell numbers at passage 1 and observed significant enrichment of cell number (#) in all systems (FIG. 13B). We observed successful growth of all three conditions by day 6 (FIG. 13C-D). When comparing Matrigel vs suspension culture over time, observed the area/size of organoids to be greater in Matrigel when compared to suspension (FIG. 13E). Bright-field (BF) images of suspension organoids reveal 3D structures, but we also observed far more debris and floating cells when compared to Matrigel (FIG. 13D). IF stains of day 6 reveal that suspension organoids are mostly COL17A1⁺ and have sporadic CRNN⁺ cells on the outside of the structure. On the other hand, Matrigel organoids were CRNN⁻ negative at this early time point (FIG. 5F), with all cells possessing COL17A1⁺. We quantified KI67⁺ cells and observe that Matrigel significantly enhanced proliferative cells when compared to suspension (P<0.05) (FIG. 13G). Finally, we evaluated whether suspension and Matrigel organoids could be grown in longer-term cultures. We observed that Matrigel provided a favorable environment for continuous 3D growth in size when compared to suspension (FIG. 13H). By day 25, Matrigel organoids had grown significantly in size and possessed a complex 3D esophageal structure with the expression of cell-type markers from all esophageal zones (FIG. 13I-J). 3D Matrigel-grown organoids show an inverse structure (basal progenitor-stem cells towards the outside vs suprabasal and differentiated cells towards the inside of the organoid) when compared to suspension organoids. It was not possible to analyze suspension organoids at the 25-day time point due to their small size and inefficient growth in this condition. These results suggest that 2D cultured cells retain their ability to form 3D organoids that can be grown in suspension or in Matrigel.

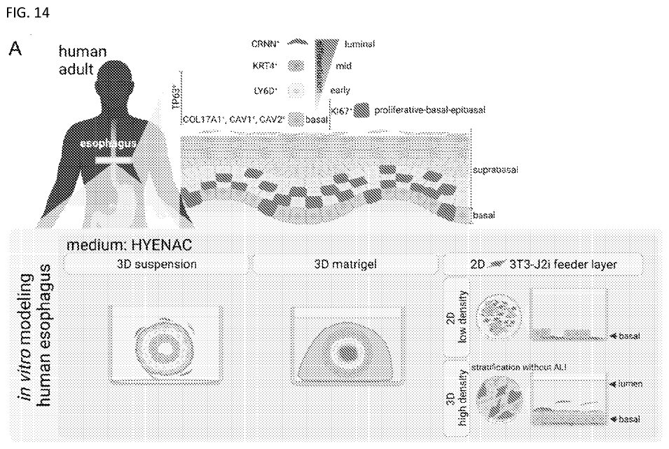

Altogether, we have created a comprehensive map of the human adult epithelium at single-cell resolution with protein validation (FIG. 14). Further, we have demonstrated and validated that 2D/3D organoids can be employed for modeling the complete epithelial layers, thereby enabling in vitro modeling of the human adult esophagus (FIG. 14).

### Example V

This example provides a discussion of Examples I-IV.

Recent advances using single-cell RNA sequencing in the human esophagus have described with more detail the epithelial subpopulations (Madissoon et al. 2019; Bogte et al. 2021). Here, we add additional data sets to this emerging body of literature, and we also validated markers from all epithelial populations predicted by scRNA-seq in tissue sections, identifying unique markers of the basal cell layer (i.e., CAV1, CAV2), and the epibasal zone (i.e LY6D), which has highest expression restricted to the second-third-fourth layer of cells. The LY6D/epibasal domain also corresponds to the domain with the highest amount of proliferation. In agreement with previous reports (Barbera et al. 2015), we find few KI67 positive cells in the basal layer. Finally, consistent with previous work, we observed that KRT4 marks early differentiating luminal cells along with more terminally differentiated cells closest to the lumen marked by CRNN. The current work is also the first to describe in vitro cultured esophageal cells using primary in vivo tissue as a benchmark. Of note, we found that the transcription factor TP63, which is known for its role in basal cell regulation in several tissues such as the skin, lungs, and esophagus in mice (Zhang et al. 2017; 2021; Domyan et al. 2011; Mou et al. 2016; Daniely et al. 2004) is not restricted to the basal zone in the human adult esophagus but is more broadly expressed throughout the basal and suprabasal zone, highlighting species-specific differences in gene/protein expression (Uhlen 2005).

While the current focus of this work was to develop and describe robust in vitro systems to study the human esophagus, we also used these methods to develop a diverse biobank of human specimens. Our long-term motivations for developing this biorepository are to better understand how genetic differences and ancestry may play a role in injury repair or disease. Multiple pediatric and adult esophageal diseases are known to have gender and racial disparities in their incidence and presentation (Hall 2020; Abraham et al. 2016; Weiler et al. 2014). For example, a recent study interrogating patients with a history of gastroesophageal reflux disease (GERD), followed a cohort of both African American (AA) and European Americans (EA) and found that 8% of EA developed the pre-malignant condition Barrett's esophagus vs, 0% of the AA did not (Alkaddour et al. 2015). These patient studies suggest that there are differences in how tissues respond to injury repair in the esophagus. Given that greater than 95% of the immortalized esophageal cell lines that have been used in the past are derived from populations of European descent (Rojas et al. 2020), enhancing the availability of diverse primary tissue will lead to an improved understanding of cell behavior, homeostasis, and disease across the human population (Rojas et al. 2020; Pepejoy and Fullerton 2016).

A key characteristic of esophageal basal stem cells is their potential to differentiate into a fully stratified tissue resembling the multi-layered in vivo structure. Numerous studies have utilized air-liquid interphase to drive in vitro esophagus cells to form a stratified and differentiated 3-dimensional tissue (Mou et al. 2016; Yamamoto et al. 2016). We also observed in the current study that these cells are capable of stratifying into an organized basal-to-luminal axis in ALI (Mou et al. 2016; Yamamoto et al. 2016). Air liquid interphase is an important technical tool for studying the epithelial cell barrier function (Kleuskens et al. 2021; Blevins et al. 2018). While ALI requires the use of transwells, it is also of interest that a similar stratified tissue, possessing basal cells and differentiated cells, can be formed by solely modifying cell density in the well of a standard tissue culture plate. Contact-inhibition of epithelial cell proliferation is well understood to drive the differentiation (Pavel et al., 2018; Eagle and Levine 1967). Such a system is straightforward and may enable scale-up for high throughput studies or studies involving the simultaneous expansion of many patient-derived cell lines. The ability to generate human esophageal 2D monolayers and 3D structures (by either high-density, suspension, and Matrigel) coupled with the ability to robustly cryopreserve and expand these cultures, means that broad distribution and implementation of these cells for different kinds of experimental assays will enable and enhance research aimed at understanding esophageal development, homeostasis, and disease. Further, these systems will be important tools to advance our understanding of how ancestral genetics, environmental factors, and carcinogens may differentially impact disease across patient populations.

While only scratching the surface, with the robust methods and benchmarking of in vitro cells to in vivo tissue using scRNA-seq and protein expression analysis, have begun to develop a biobank of esophageal cell lines from across the diversity of the human population, and from a wide range of healthy and diseased states (Table 1). In vitro cellular models of the human esophagus can therefore be used to assess stem cell homeostasis as well as the effects of damaging and carcinogenic agents in patient-derived cells across the spectrum of diversity in the human population.

### Example VI

This example provides the materials and methods implemented in performing the experiments recited in Examples I-V.

### Sample Collection

Histologically normal biopsies of the esophageal squamous epithelium were collected from consenting men and women who underwent upper endoscopy or surgical resection between 2017 and 2020 at the time of scheduled inflammatory bowel disease (IBD) or BE screening or tumor surgical resection at the University of Michigan Health System. Samples were collected using protocols approved by the University of Michigan institutional review board (IRB). Fresh samples were collected on cold HYENAC, and either used immediately for single-cell dissociation followed by scRNA-seq or were processed for culture; otherwise, biopsies were cryopreserved and stored at −80 C until use, at which point they could be thawed and processed for generating new culture lines. To cryopreserve biopsies, we mince tissue into fine pieces and freeze using 1 ml/vial of HYENAC+20% CBS serum and 10% DMSO. Cryovials are placed in Mr. Frosty's at −80 overnight and moved to liquid nitrogen tanks for long-term preservation. For culturing cell lines, we found that fresh biopsies could be processed immediately and grown at a 100% success rate. We observed that biopsy samples in HYENAC medium can be kept for 24 hrs at 4 C and can be grown successfully. Further, biopsies can be shipped cold overnight (or cryopreserved with 10% DMSO), at which point viable cell lines could still be robustly established. For access to detailed protocols see https://www.umichttml.org/protocols. Cell turnover was calculated using the previously described method (Sherley, Stadler, and Stadler 1995). All in vitro cultures were generated in the lab and have a negative test for mycoplasma.

### Patient Samples Information

The patient's race was self-identified. For white non-Hispanics (W-NH), we used the nomenclature European American (EA); for Black, we used African American (AA). Biological replicates utilized for single-cell RNA sequencing came from the normal squamous biopsies.

### Tissue Processing and Staining

Patient biopsy/tissues were fixed in 4% paraformaldehyde (Sigma) overnight, washed with PBS, and then dehydrated in an alcohol series: 30 minutes each in 25%, 50%, 75% methanol: PBS/0.05% Tween-20, followed by 100% methanol, 100% ethanol, and 70% ethanol. Tissue was processed into paraffin using an automated tissue processor (Leica ASP300). Paraffin blocks were sectioned seven μM thick, and immunohistochemical staining was performed as previously described, (Spence et al. 2011). Briefly, slides were rehydrated in a series of HistoClear, 100% ethanol, 95% ethanol, 70% ethanol, 30% ethanol, and Dand I H2O with two changes of 3 minutes each. Antigen retrieval was performed in 1× sodium citrate buffer in a vegetable steamer for 40 minutes. Following antigen retrieval, slides were washed in PBS and permeabilized for 10 minutes in 0.1% TritonX-100 in 1×PBS, blocked for 45 minutes in 0.1% Tween-20, 5% normal donkey serum. Antibodies used in this study can be found in the Key Resources Table. Primary antibodies were diluted in the blocking solution and applied overnight at 4° C. Slides were then washed three times in 1×PBS. Secondary antibodies and DAPI were diluted in the blocking solution and applied for 60 minutes at room temperature. Slides were then washed three times in 1×PBS and coverside with ProLong Gold. For a detailed list of antibodies and conditions.

### Hematoxylin and Eosin

According to the manufacturer's instructions, H&E staining was performed using Harris Modified Hematoxylin (FisherScientific) and Shandon Eosin Y (ThermoScientific).

### Imaging and Image Processing

Fluorescently stained slides were imaged on a Nikon A-1 confocal microscope. Brightness and contrast adjustments were carried out using ImageJ (National Institute of Health, USA),and adjustments were made uniformly across images. For FIG. 10E, three images of DAPI and KRT4 were taken at the same exposure for each patient (n=3). Photos were uploaded to FUI, and full image pixel intensity was quantified for DAPI and KRT4. Pixel intensity was normalized to their match DAPI imaged and plotted as a ratio KRT4/DAPI.

For FIG. 5 3-dimensional growth, bright field images (n=3-4 photos per well) over daytime points were taken. Open-source image analysis software FIJI was used to measure the area of all organoids per image. For FIG. 11B-C, FIJI was utilized to determine basal vs. luminal using orthogonal view and maximum intensity projection. Z-stack images were open in FUI—then Image>Stacks>reslice followed by Image>stack>z-project (FIG. 11B-C).

### Schematics and Diagrams

Schematics made with BioRender, 2021, (Suite 2021), and Illustrator.

### Quantification and Statistical Analysis

Statistical analyses and plots were generated in Prism 8 software (GraphPad). For all statistical tests, a significance value of 0.05 was used. For every analysis, the strength of P values is reported in the figures according to the following: P>0.05, *P≤0.05, **P≤0.01, ***P≤0.001, ****P≤0.0001. Details of statistical tests can be found in the figure legends. Except for scRNA-seq, three HT lines were used across experiments with at least 2-3 independent experiments and at least 2-3 technical replicates per experiment.

### Tissue Dissociation for Single Cell RNA Sequencing

To dissociate patient biopsies for single-cell RNA sequencing, tissue was placed in a petri dish with ice-cold 1X HBSS (with Mg2+, Ca2+). To prevent adhesion of cells, all tubes and pipette tips were pre-washed with 1% BSA in 1×HBSS. The tissue was minced manually using spring-squeeze scissors before being transferred to a 15 mL conical containing 1% BSA in HBSS. Tubes were spun down at 500 G for 5 minutes at 10° C., after which excess HBSS was aspirated. Mix 1 from the Neural Tissue Dissociation Kit (Miltenyi, 130-092-628) containing dissociation enzymes and reagents was added and incubated at 10° C. for 15 min. Mix 2 from the Neural Tissue Dissociation Kit was added, and the suspension was fluxed through P1000 pipette tips, interspersed with 10 min incubations at 10° C. Flux steps were repeated as needed until cell clumps were no longer visible under a stereomicroscope. Cells were filtered through a 1% BSA-coated 70 μm filter using 1×HBSS, spun down at 500 g for 5 minutes at 10° C., and resuspended in 500 μl 1×HBSS (with Mg2+, Ca2+). 1 mL of RBC Lysis Buffer (Roche, 11814389001) was added, and tubes were incubated on a rocker at 4° C. for 15 minutes. Cells were spun down at 500 G for 5 minutes at 10° C., then washed twice in 2 mL 1% BSA, being spun down at 500 G for 5 minutes at 10° C. each time. A hemocytometer was used to count cells, then spun down and resuspended to reach a concentration of 1000 cells/μL and kept on ice.

### Single-Cell Library Preparation

The 10× Chromium at the University of Michigan Advanced Genomics Core facility was then used to create single-cell droplets with a target of capturing 5,000-10,000 cells. The Chromium Next GEM Single Cell 3′ Library Construction Kit v3.1 prepared single-cell libraries according to manufacturer instructions.

### Sequencing Data Processing and Cluster Identification

The University of Michigan Advanced Genomic Core Illumina Novaseq performed all single-cell RNA sequencing. Gene expression matrices were constructed from raw data by the 10× Genomic Ranger with the human reference genome (hg19). The Single Cell Analysis for Python was utilized for analysis as previously described by (Wolf, Angerer, and Theis 2017). Filtering parameters for gene count range, unique molecular identifier (UMI) counts, and mitochondrial transcript fraction was implemented for each data set to verify high-quality input data. All tissue data sets were combined after organ-specific quality filtering had been performed for the remainder of the processing. Highly variable genes were removed, gene expression levels were log normalized, and effects of UMI count and Mitochondrial transcript function variations were regressed via linear regression. Z-transformation was then performed on gene expression values before samples were again separated by organ for downstream analysis. The UMAP algorithm (Becht et al. 2019; McInnes, Healy, and Melville 2018) was utilized alongside Louvain algorithm cluster identification within Scanpy with a resolution of 0.6 (Blondel et al. 2008) to perform a graph-based clustering of the top 10-11 principal components. A detailed protocol for tissue dissociation for single-cell RNA seq can be found at www.jasonspencelab.com/protocols.

### Computational Analysis of Single-Cell RNA Sequencing Data

### Overview

To visualize distinct cell populations within the single-cell RNA sequencing dataset, we employed the general workflow outlined by the Scanpy Python package (Wolf, Angerer, and Theis 2017). This pipeline includes the following steps: filtering cells for quality control, log normalization of counts per cell, extraction of highly variable genes, regressing out specified variables, scaling, reducing dimensionality with principal component analysis (PCA), and uniform manifold approximation and projection (UMAP) (Becht et al. 2019), and clustering by the Louvain algorithm (Blondel et al. 2008).

Sequencing Data and Processing FASTQ Reads into Gene Expression Matrices.

All single-cell RNA sequencing was performed at the University of Michigan Advanced Genomics Core with an Illumina Novaseq 6000. The 10× Genomics Cell Ranger pipeline was used to process raw Illumina base calls (BCLs) into gene expression matrices. BCL files were demultiplexed to trim adaptor sequences and unique molecular identifiers (UMIs) from reads. Each sample was then aligned to the human reference genome (hg19) to create a filtered feature bar code matrix that contains only the detectable genes for each sample.

### Quality Control

To ensure the quality of the data, all samples were filtered to remove cells expressing too few or too many genes (FIG. 1/FIG. 2/FIG. 3/FIG. 4/FIG. 5/FIG. 8/FIG. 6: <500, >7500, or a fraction of mitochondrial genes greater than 0.2.

### Normalization and Scaling

Data matrix read counts per cell were log normalized, and highly variable genes were extracted. Using Scanpy's simple linear regression functionality, the effects of total reads per cell and mitochondrial transcript fraction were removed. The output was then scaled by a z-transformation. Following these steps, a total of (FIG. 1—9039 cells, 3897 genes; FIG. 2/FIG. 3 (extracted)—7796 cells, 2651 genes; FIG. 4/FIG. 5A-C—10550 cells, 4269 genes; FIG. 5E-F—HT239(4617 cells, 4845 genes), HT344 (895 cells, 3034 genes), HT328 (4133 cells, 5195 genes), FIG. 8C-H—7389 cells, 3413 genes, FIG. 6—19589 cells, 3486 genes.

### Variable Gene Selection

Highly variable genes were selected by splitting genes into 20 equal-width bins based on log normalized mean expression. Normalized variance-to-mean dispersion values were calculated for each bin. Genes with log normalized mean expression levels between 0.125 and 3 and normalized dispersion values above 0.5 were considered highly variable and extracted for downstream analysis.

### Batch Correction

We have noticed batch effects when clustering data due to technical artifacts such as data acquisition timing or dissociation protocol differences. To mitigate these effects, we used the Python package BBKNN (batch balanced k nearest neighbors) (Polaiski et al. 2020). BBKNN was selected over other batch correction algorithms due to its compatibility with Scanpy and optimal scaling with large datasets. This tool was used in place of Scanpy's nearest neighbor embedding functionality. BBKNN uses a modified procedure to the k nearest neighbors' algorithm by first splitting the dataset into batches defined by technical artifacts. For each cell, the nearest neighbors are then computed independently per batch rather than finding the nearest neighbors for each cell in the entire dataset. This helps form connections between similar cells in different batches without altering the PCA space. After completion of batch correction, cell clustering should no longer be driven by technical artifacts.

### Dimension Reduction and Clustering

Principal component analysis (PCA) was conducted on the filtered expression matrix followed. Using the top principal components, a neighborhood graph was calculated for the nearest neighbors FIG. 1—16 principal components, 30 neighbors; FIG. 2/FIG. 3—9 principal components, 11 neighbors; FIG. 4/FIG. 5—30 principal components, 16 neighbors; FIG. 8C-H—11 principal components, 15 neighbors; FIG. 6—16 principal components, 30 neighbors. BBKNN was implemented when necessary and calculated using the top 50 principal components with three neighbors per batch. The UMAP algorithm was then applied for visualization on two dimensions. Using the Louvain algorithm, clusters were identified with a resolution of (FIG. 1—0.3; FIG. 2/FIG. 3—0.2; FIG. 4/FIG. 5—0.3; FIG. 8—0.4, and FIG. 6—0.3).

### Cluster Annotation

Each cluster's general cell identity was annotated using canonically expressed gene markers. Markers utilized include epithelium (CDH1), mesenchyme (VIM), neuronal (POSTN, S100B, STMN2, ELAV4), endothelial (ESAM, CDH5, CD34, KDR), and immune (CD53, VAMP8, CD48, ITGB2).

### Sub-Clustering

After annotating clusters within the UMAP embedding, specific clusters of interest were identified for further sub-clustering and analysis. The corresponding cells were extracted from the original filtered but unnormalized data matrix to include (FIG. 2A/3—9039 cells, 3897 genes). The extracted cell matrix underwent log normalization, variable gene extraction, linear regression, z transformation, and dimension reduction to obtain a 2-dimensional UMAP embedding for visualization.

### 3D In Vitro Modeling

### Air Liquid Interphase

On Day 0, 200,000 cells were seeded into transwells. HYENAC medium was kept in both chambers for 72 hrs after seeding. The medium on the upper chamber was removed three days post-seeding to create the air-liquid interphase, as previously described. To measure TEER, the EVOM2 epithelial voltohmmeter was employed (World Precision Instruments). The medium was briefly added to the apical chamber for ALI cultures and then removed following measurements. On day 14, wells were either fixed, dehydrated, embedded into paraphing, section, and stained for H&E. Or, in well, protein staining was performed, using 100% methanol fixation for 20 mins at −20° C. Followed by two washes of PBS and can be stored at 4 C until immunofluorescence. On the day of Immunofluorescence, wells are then incubated with 10% Neural Buffer Formalin (20 mins), washed with tris-buffered saline (TBS) solution for 5 mins, incubated with 100% cold methanol for 5 mins, washed with TBS for 20 mins, incubated with a blocking solution (composed of 1% BSA, 5% Donkey serum, and 0.2% of Triton X in TBS) for 1 hr. Primary antibodies (diluted in TBS solution with 1% BSA) were added, and plates were incubated overnight at 4° C. in a humidifier chamber. Primary antibodies were used at a concentration of 1:500, except for Hu-Nu, which was used at 1:200. Wash with TBS-T 3× times for 5 mins. Incubate with respective fluorescence secondary antibodies (1:500) for 1 hr. Wash 3× times with TBS-T for 5 mins. On a microscope slide, add a droplet of 5-10 uL of prolong gold (trying to minimize bubble formation). Carefully remove the transwell from the plate with a scalpel, carefully detach the transmembrane from the transwell with tweezers, and carefully place the transmembrane into a microscope slide, making sure the side with cells faces and is put in contact with the glass of the slide. They were stored at 4 C/−20° C. until ready for imaging with the confocal microscope.

### Stratification Using Cell Density

On day 0, Siliconized Glass Circle Cover Slides [Cat. #HR3-277 (12 mm), Hampton Research] were added into each well of a 24-well plate and plates were pre-coated with ˜18,000-20,0000 cells of 3T3-J2i in 500 uL/well (DMEM+20% CBS). On day 1, cells were trypsinized, counted, resuspended, and plated at 50-125,000 cells/well (low density) and 500,000 cells/well (high density). The medium was changed every two days. On day five or greater (depending on the doubling life of each cell line), cells were washed with 500 uL/well of PBS and fixed with 500 uL/well 100% cold methanol for 20 mins at −20° C. Subsequently, cells were briefly washed three times with 500 uL/well PBS and stored with 500 uL/well PBS at 4° C. until ready for immunofluorescent staining as described above. (Visual schematic summary of the protocol can be found in FIG. 11A).

### Matrigel®

On day 0, 2D expanded cells were trypsinized, counted, resuspended, and plated at 50 to 125,000 cells/well into Matrigel® (REF #356234, Matrigel® Matrix, Corning Inc.). In short, cells are spun and embedded in Matrigel® droplets, then incubated for 10-15 mins to solidify Matrigel®. Media changes every 2-3 days until sample collection and processing. For detailed information on Matrigel® protocols, please refer to: https://www.corning.com/worldwide/en/products/life-sciences/products/surfaces/matrigel-matrix.html.

### Suspension

Suspension plates were made as previously described (Capeling et al., 2022). On day 0, 2D expanded cells were trypsinized, counted, resuspended, and plated at 50 to 125,000 cells/well into suspension plates. Media changes every 2-3 days until sample collection and processing.

### Example VII

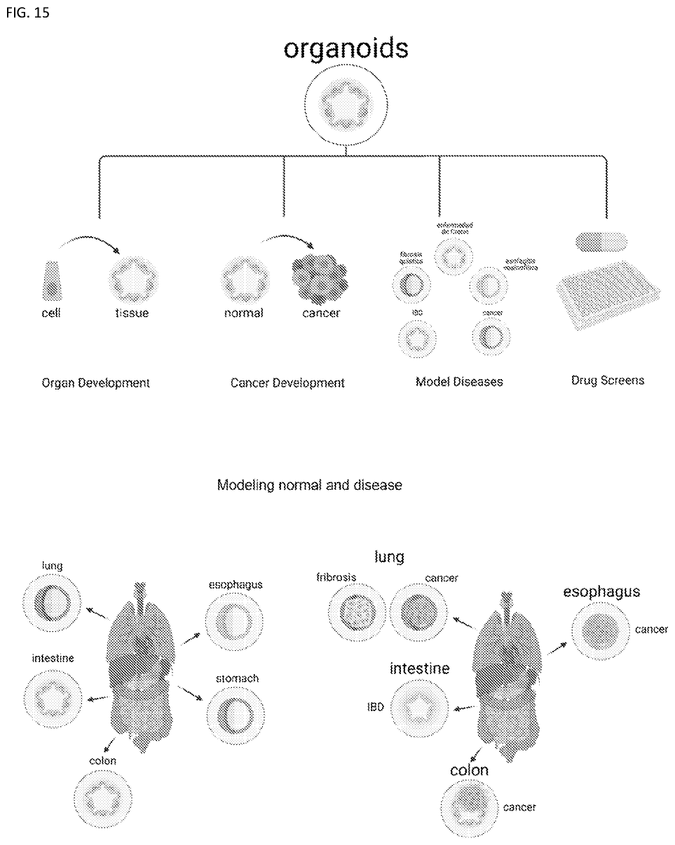

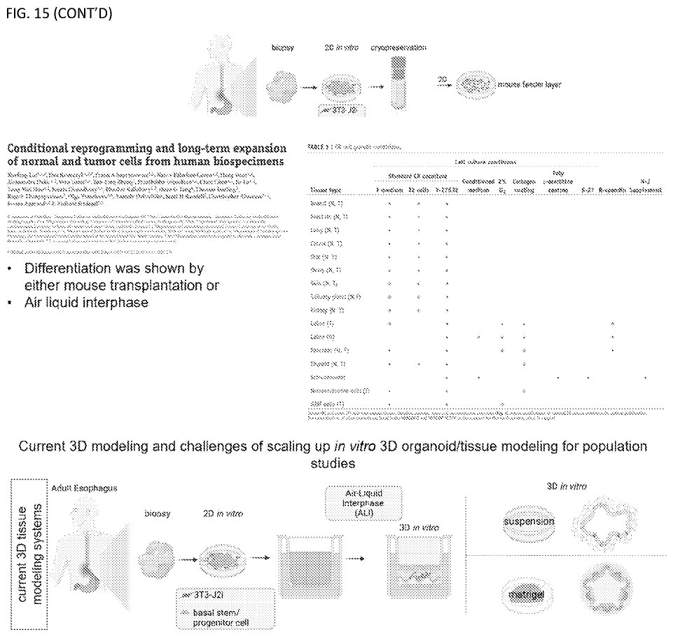

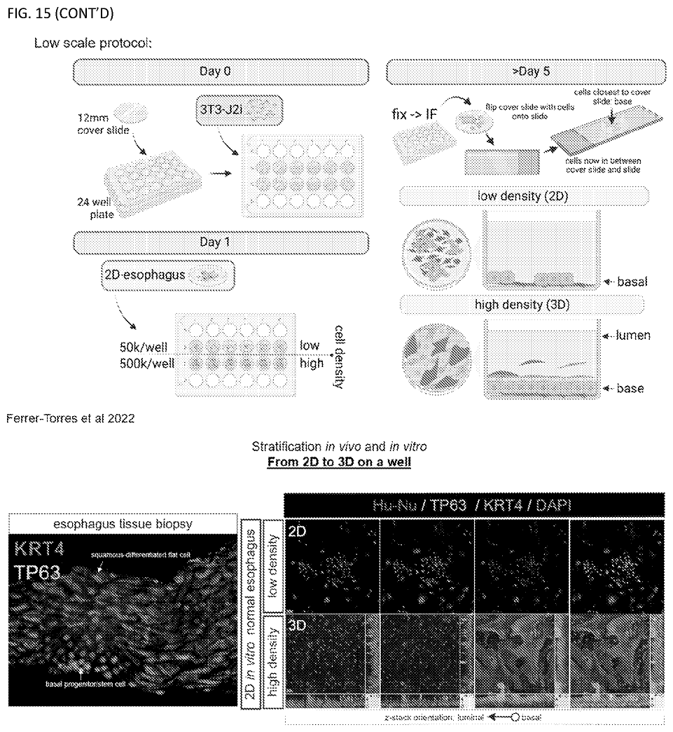

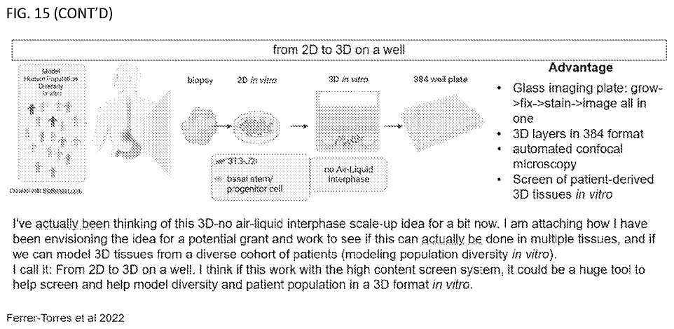

FIG. 15 provides clinical characteristics of primary human esophageal samples for 2D or 3D in vitro culture.

### Example VIII. Media Culture Conditions for Primary Esophageal Tissue-Cells In Vitro Growth

Procedure for the culture of primary esophageal cells culture from biopsies or surgical specimens.

### General Description:

Prior art: Previous work has attempted to culture healthy and diseased esophagus epithelial cells from human patients. However, these previous studies have been limited in different ways: 1. They showed only short-term culture was possible; 2. They did not adequately disclose media composition that was used precluding reproduction of the studies (i.e. https://www.nature.com/articles/ncomms10380); 3. Studies were performed in mouse, but not human (i.e. https://pubmed.ncbi.nlm.nih.gov/27320041/) 4. The esophagus is considered to be a “stratified epithelium”. Prior studies did not specifically identify a media cocktail that allowed the long-term growth and expansion of esophageal stem cells and their progeny.

### Current Work:

The current work describes a unique media combination for long-term culture of adult and fetal human esophageal cells, including esophageal stem cells and differentiated cells. This method seeds esophagus epithelial cells onto sub-lethally irradiated feeder cells (3T3-J2i) (https://pubmed.ncbi.nlm.nih.gov/1052771/plus a media that provides the following: a rho-kinase inhibitor (i.e. Y-27632), plus a WNT-activator/agonist (i.e. CHIR99021), plus EGF, plus an inhibitor of TGFβ signaling (i.e. A-8301) and an inhibitor of BMP signaling (i.e. NOGGIN). Finally, the media contained hydrocortisone.

Using this unique media, we showed the following:
1. The ability to cryopreserve fresh human esophagus tissue (i.e. biopsies), which could later be thawed in order to make cell lines or organoids.
2. Esophagus cell lines established in this media possessed esophagus stem cells and differentiated esophageal cells, which could be maintained in long-term culture (i.e. many generations), could be frozen and thawed.
3. Esophagus cells maintained using this media could be cultured with or without feeder cells, could be cultured in a 2-dimensional format (i.e. on a tissue culture dish or a glass cover-slip), or in a 3-dimensional format (i.e. organoids, spheroids).

A detailed methodology and protocol can be found below.

### Materials

Surgical instruments: scalpels, forceps
BSA-coated plastic ware to prevent tissue/crypts from sticking to surfaces. Incubate in 0.1% BSA in DPBS (Sigma A8806; sterile-filtered) for 2-30 min at room temp; store at 4° C.
   BSA-coat 30 minutes: 200 μl and 1000 μl tips; 2 mL tubes, 100 mm petri dish

### Reagents

10 μM Y-27632 (Tocris; 125410): 2.5 mM stock in H₂O. Serum-free cultures can use 5 μM. Presumed sterile, not filtered. Stability: aliquots at −20° C. for up to 1 yr.
2.5 μM CHIR99021 (Tocris; 4423; at establishment/passaging; 10 mM stock in DMSO (−80° C.). Working solutions used within the day (1:4000)
A 83-01 (R&D Tocris, 2939): TGF-β inhibitor, 1 mM stock in DMSO at −80° C. Working solutions used within the day; dilute into warm media at 0.5 μL/mL for 500 nM final.
Calf Serum (CBS; ATCC 30-2030)
Hydrocortisone (Sigma H0888): To prepare 330 μg/mL stock solution: Add 10 mg/10 mL absolute ethanol; gently swirl to dissolve: then dilute 3.3 ml of above solution into 6.7 mL EtOH for final stock 330 μg/mL
Noggin: recombinant human noggin (RnD; 6057-ng; BSA carrier); stock 100 μg/mL (Sha has ‘10×’ 1 mg/mL); final 100 ng/mL; predicted molecular mass 23 kDa
EGF (236-EG-01M; $409; R&D)
   1 mg into 10 mL sterile room temperature PBS (with 0.1% BSA)
   250 μL/vial; 1000×; 40 vials
   3 month expiration at −20 to −80 C (R&D); but Sigma states at least 2 years −20° C.
   (0.1% BSA)
   1 month, 2 to 8° C. under sterile conditions after reconstitution (R&D and Sigma).
   Final Concentration: 100 ng/mL
Basal Mediums:
   Advanced DMEM/F-12 (Invitrogen 12634028): add 2 mM GlutaMax, 10 mM HEPES
   Phenol Red-free DMEM/F-12 (Sigma D2906; formulation)
      Contains L-glutamine (1 month expiration) and 15 mM HEPES
      WITHOUT sodium bicarbonate, phenol red, AlbuMAX, ascorbic acid, insulin & transferrin (in N2/B27)
      ADD 1.2 g/L NaHCO₃
Basal Medium Plus:
   Add to basal mediums: 1×N-2 media supplement, 1×B-27 Supplement Minus Vitamin A, 1 mM N-Acetyl-L-cysteine, 100 ng/mL huEGF (R&D 236-EG-01M), 100 μg/ml Primocin (InvivoGen; #ant-pm-1)
   25 μg/ml gentamicin (½×) & 2.5 μg/ml amphotericin (Gibco 15290018): first 48 hrs and cryopreservation medium:
   3T3-J2 irradiated cells from Tasha of Colacino lab thawed by adding to 24 mL warm 3T3 medium: one vial of 450,000 cells into 2×9 wells of 24-well plate (11:15 AM); froze 2 vials in Dewar 3, canister 3, canes 2

| | | | Hydrocortisone | | | | | |
|---|---|---|---|---|---|---|---|---|
| Media | Above | Y27632 | 0.33 μg/mL | EGF | A83-01 | Noggin | CHIR99021 | CBS |
| Condition | Basal | 10 μm | 0.9 μM | 100 ng/mL | 500 nM | 100 ng/mL | 2.5 μM | 20% |
| #1-HYENA | + | + | + | + | + | + | | |
| #4- | + | + | + | + | + | + | + | + |
| HYENAC + | | | | | | | | |
| CBS | | | | | | | | |

Mr. Frosty Cryopreservation Container: https://www.fishersci.com/shop/products/thermo-scientific-mr-frosty-freezing-container-nalgene-mr-frosty-freezing-container-1-2 ml-cryogenic-tubes-pc-clear-w-blue-lid-1-cs/1535050

Procedure A—Cryopreserving and/or Shipping Live Biopsies
I. Cryopreserving Cells from Primary Biopsy
Note: Process biopsies on ice
a) Collect biopsies or surgical specimens into 1 mL cold #1 HYENA media (Medium A; HYENA media+Fungizone)
   in Eppendorf tubes.
a) Place biopsy in dish. Remove residual medium. Finely cut individual biopsies into very small pieces (7 pt pen spot; minced) using spring-loaded scissors or a scalpel. Smaller is better.
b) Resuspend cut biopsy pieces in 900 μL of medium #4 (Medium B; HYENAC+CBS).
c) Transfer to cryopreservation vial.
d) Add 100 μL DMSO to vial (10% DMSO cryopreservation media)
e) Gently invert vial 3 times.
f) Freeze using a Mr. Frosty (or equivalent) for 24 hrs at −80 (or until shipment time).
g) Move to Liquid Nitrogen for long term preservation

### II. Shipping Live Biopsies

b) Place fresh biopsy specimen into to 1 mL #1 HYENA media (Medium A; HYENA media+Fungizone) in a cryopreservation vial or 1.5 mL microcentrifuge tube. If using microcentrifuge tube, wrap with parafilm.
c) Keep cold by placing on wet ice or using cold packs.
d) Ship on wet ice or using cold packs.
   Procedure B—Generating Primary Esophageal Cell Lines from Human Biopsies
A. Plate Preparation (*24 hrs, 4 hrs, or 1 hr in advance to preparing the tissue)
   A. Add 100,000 cells per well (6-well plate) 24 hrs (or 4 hrs), before sample collection. (This allows for the J2i to adhere and form a matrix that will allow the esophagus cells to attach to the plate).
   B. for non-J2s plating, coat plate with matrigel (1 hr matrigel incubation at RT)
B. Tissue Processing: on ice
   1) Collect biopsies or surgical specimens into HYENA media conditions.
   2) Minced on BSA-coated petri dish on ice, minced samples into tiny pieces.
   3) Resuspend minced cells in 1,200 uL of medium #4 (HYENAC+CBS). Added 200 uL to each well, which was prepped with 1,500 uL of media #4 (HYENAC+CBS). Total 450 μL. Observed numerous single cells with intact membranes. (FIG. 1A)
C. Passaging cells
   i. 24 hrs before passaging, add 100,000 J2i into each well of a 6-well plate, with 2,000 ul of DMEM+10% CBS/well (incubate for 24 hrs, or at least 4 hrs, before passaging the esophageal cells)
   ii. Add 500 ul of trypsin EDTA to each well of esophagus culture cells. (1-3 min incubation)
   iii. Inactivated trypsin adding 2 mls of DMEM+10% CBS media.
   iv. Resuspend cells in media and transfer to a 15 ml tube
   v. Spin cells @500 g for 5 mins
   vi. Remove media, leaving pellet of cells on the bottom
   vii. Resuspend pellet of cells into 1,200 uL of medium #4 (HYENAC+CBS). Add 200 uL off cells suspension to each well, which was prepped with 1,500 uL/well of media #4 (HYENAC+CBS).
*long exposure to trypsin may cause the esophagus cells to lose their adherence capabilities.
**esophagus cells differentiated when confluency is reach. Split-passage cells at about 70-80% confluency to prevent differentiation.
D. Freezing cells
   i. Add 500 ul of trypsin EDTA to each well of esophagus culture cells. (1-3 min incubation)
   ii. Inactivated trypsin adding 2 mls of DMEM+10% CBS media.
   iii. Resuspend cells in media and transfer to a 15 ml conical
   iv. Spin cells @500 g for 5 mins
   v. Remove media, leaving pellet of cells on the bottom
   vi. Resuspend pellet of cells into 5,000 uL of medium #4 (HYENAC+CBS). Add 10% DMSO
   vii. Add 1 ml of suspension of cells into cryovials (approximately 5 vials per freeze of a 6 well plate).
   viii. Freeze vials using cryopreservation Mr. Frosty freezing container
   ix. Place in −80 C o/n.
   x. Move vials intro liquid nitrogen tank within a 24 hrs span of cryopreservation.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes. Complete citations for the references cited within the application are provided within the following reference list. Indeed, each of the following references are herein incorporated by reference in their entireties:
Abraham, Albin, Seth Lipka, Rabab Hajar, Bhuma Krishnamachari, Ravi Virdi, Bobby Jacob, Prakash Viswanathan, and Paul Mustacchia. 2016. “Erosive Esophagitis in the Obese: The Effect of Ethnicity and Gender on Its Association.” Gastroenterology Research and Practice 2016. https://doi.org/10.1155/2016/7897390.
Alkaddour, Ahmad, Camille McGaw, Rama Hritani, Carlos Palacio, Rahman Nakshabendi, Juan Carlos Munoz, and Kenneth J. Vega. 2015. “African American Ethnicity Is Not Associated with Development of Barrett's Oesophagus after Erosive Oesophagitis.” Digestive and Liver Disease 47 (10): 853-56. https://doi.org/10.1016/j.dld.2015.06.007.
Arnold, Melina, Mathieu Laversanne, Linda Morris Brown, Susan S Devesa, and Freddie Bray. 2017. “Predicting the Future Burden of Esophageal Cancer by Histological Subtype: International Trends in Incidence up to 2030.” Am J Gastroenterol. American College of Gastroenterology. http://dx.doi.org/10.1038/ajg.2017.155.
Bailey, Dominique D., Yongchun Zhang, Benjamin J. van Soldt, Ming Jiang, Supriya Suresh, Hiroshi Nakagawa, Anil K. Rustgi, Seema S. Aceves, Wellington V. Cardoso, and Jianwen Que. 2019. “Use of HPSC-Derived 3D Organoids and Mouse Genetics to Define the Roles of YAP in the Development of the Esophagus.” Development (Cambridge) 146 (23): 1-11. https://doi.org/10.1242/dev.178855.
Barbera, Mariagnese, Massimiliano D. Pietro, Elaine Walker, Charlotte Brierley, Shona MacRae, Benjamin D. Simons, Phil H. Jones, John Stingl, and Rebecca C. Fitzgerald. 2015. “The Human Squamous Oesophagus Has Widespread Capacity for Clonal Expansion from Cells at Diverse Stages of Differentiation.” Gut 64 (1): 11-19. https://doi.org/10.1136/gutjnl-2013-306171.
Becht, Etienne, Leland McInnes, John Healy, Charles Antoine Dutertre, Immanuel W. H. Kwok, Lai Guan Ng, Florent Ginhoux, and Evan W. Newell. 2019. “Dimensionality Reduction for Visualizing Single-Cell Data Using UMAP.” Nature Biotechnology 37 (1): 38-47. https://doi.org/10.1038/nbt.4314.
Blevins, Christopher H., Prasad G. Iyer, Marcelo F. Vela, and David A. Katzka. 2018. “The Esophageal Epithelial Barrier in Health and Disease.” Clinical Gastroenterology and Hepatology 16 (5): 608-17. https://doi.org/10.1016/j.cgh.2017.06.035.
Blondel, Vincent D., Jean Loup Guillaume, Renaud Lambiotte, and Etienne Lefebvre. 2008. “Fast Unfolding of Communities in Large Networks.” Journal of Statistical Mechanics: Theory and Experiment 2008 (10): 1-12. https://doi.org/10.1088/1742-5468/2008/10/P10008.
Bogte, Auke, Harry Begthel, Lodewijk A A Brosens, Hans Clevers, Georg A Busslinger, Bas L A Weusten, Auke Bogte, Harry Begthel, and Lodewijk A A Brosens. 2021. “Human Gastrointestinal Epithelia of the Esophagus, Stomach, and Duodenum Resolved at Single-Cell Resolution L1 L1 Human Gastrointestinal Epithelia of the Esophagus, Stomach, and Duodenum Resolved at Single-Cell Resolution.” CellReports 34 (10): 108819. https://doi.org/10.1016/j.celrep.2021.108819.
Capeling, Meghan M., Sha Huang, Charlie J. Childs, Joshua H. Wu, Yu Hwai Tsai, Angeline Wu, Neil Garg, et al. 2022. “Suspension Culture Promotes Serosal Mesothelial Development in Human Intestinal Organoids.” Cell Reports 38 (7): 110379. https://doi.org/10.1016/j.celrep.2022.110379.
Co, Julia Y, Mar Margalef-catala, Xingnan Li, Amanda T Mah, Calvin J Kuo, Denise M Monack, Manuel R Amieva, et al. 2019. “Controlling Epithelial Polarity: A Human Enteroid Model for Host-Pathogen Interactions Resource Controlling Epithelial Polarity: A Human Enteroid Model for Host-Pathogen Interactions,” 2509-20. https://doi.org/10.1016/j.celrep.2019.01.108.
Daniely, Yaron, Grace Liao, Darlene Dixon, R. Ilona Linnoila, Adriana Lori, Scott H. Randell, Moshe Oren, and Anton M. Jetten. 2004. “Critical Role of P63 in the Development of a Normal Esophageal and Tracheobronchial Epithelium.” American Journal of Physiology—Cell Physiology 287 (1 56-1): 171-81. https://doi.org/10.1152/ajpcell.00226.2003.
DeWard, Aaron D., Julie Cramer, and Eric Lagasse. 2014. “Cellular Heterogeneity in the Mouse Esophagus Implicates the Presence of a Nonquiescent Epithelial Stem Cell Population.” Cell Reports 9 (2): 701-11. https://doi.org/10.1016/j.celrep.2014.09.027.
Domyan, Eric T, Elisabetta Ferretti, Kurt Throckmorton, Yuji Mishina, Silvia K Nicolis, and Xin Sun. 2011. “Signaling through BMP Receptors Promotes Respiratory Identity in the Foregut via Repression of Sox2.” Development (Cambridge, England) 138 (5): 971-81. https://doi.org/10.1242/dev.053694.
Doupé, David P, Maria P Alcolea, Amit Roshan, Gen Zhang, Allon M Klein, Benjamin D Simons, Philip H Jones, et al. 2012. “A Single Progenitor Population Switches Behavior to Maintain and Repair Esophageal Epithelium.” Science 337 (6098): 1091-93. https://doi.org/10.1126/science.1218835.
Eagle, Harry, and Elliot M Levine. 1967. “Growth Regulatory Effects of Cellular Interaction.” Nature 72 (138): 1102-6. https://doi.org/10.1038/2131102a0.
El-Serag, H B and Sonnenberg, A. 1997. “Associations between Di V Erent Forms of Gastro-Oesophageal Reflux Disease,” no. Icd: 594-99.
El-Serag, H. B., S. Sweet, C. C. Winchester, and J. Dent. 2014. “Update on the Epidemiology of Gastro-Oesophageal Reflux Disease: A Systematic Review.” Gut 63 (6): 871-80. https://doi.org/10.1136/gutjnl-2012-304269.
El-Serag, Hashem B., Nancy J. Petersen, Junaia Carter, David Y. Graham, Peter Richardson, Robert M. Genta, and Linda Rabeneck. 2004. “Gastroesophageal Reflux among Different Racial Groups in the United States.” Gastroenterology 126 (7): 1692-99. https://doi.org/10.1053/j.gastro.2004.03.077.
Ernst, Linda M, Eduardo D Ruchelli, Chrystalle Katte Carreon, and Dale S Huff. 2019. Color Atlas of Human Fetal and Neonatal Histology. Color Atlas of Human Fetal and Neonatal Histology. Cham: Springer International Publishing AG. https://doi.org/10.1007/978-3-030-11425-1.
Fleischer, David E, Bergein F Overholt, Virender K Sharma, Alvaro Reymunde, Michael B Kimmey, Ram Chuttani, Kenneth J Chang, et al. 2008. “Endoscopic Ablation of Barrett's Esophagus: A Multicenter Study with 2.5-Year Follow-Up.” Gastrointestinal Endoscopy 68 (5): 867-76. https://doi.org/10.1016/j.gie.2008.03.008.
Hall, Gregory L. 2020. Patient-Centered Clinical Care for African Americans. Patient-Centered Clinical Care for African Americans. https://doi.org/10.1007/978-3-030-26418-5.
Houben, C. H. and Curry, J. I. 2008. “Current Status of Prenatal Diagnosis, Operative Management and Outcome of Esophageal Atresia/Tracheo-Esophageal Fistula.” Prenatal Diagnosis 28: 667-675. https://doi.org/10.1002/pd.1938.
Kasagi, Yuta, Prasanna M. Chandramouleeswaran, Kelly A. Whelan, Koji Tanaka, Veronique Giroux, Medha Sharma, Joshua Wang, et al. 2018. “The Esophageal Organoid System Reveals Functional Interplay Between Notch and Cytokines in Reactive Epithelial Changes.” Cmgh 5 (3): 333-52. https://doi.org/10.1016/j.jcmgh.2017.12.013.
Kim, Jihun, Reanne Bowlby, Andrew J. Mungall, A. Gordon Robertson, Robert D. Odze, Andrew D. Cherniack, Juliann Shih, et al. 2017. “Integrated Genomic Characterization of Oesophageal Carcinoma.” Nature. https://doi.org/10.1038/nature20805.
Kleuskens, Mirelle T. A., Maria L. Haasnoot, Bart M. Herpers, Marleen T. J. van Ampting, Albert J. Bredenoord, Johan Garssen, Frank A. Redegeld, and Betty C. A. M. van Esch. 2021. “Butyrate and Propionate Restore Interleukin 13-Compromised Esophageal Epithelial Barrier Function.” Allergy: European Journal of Allergy and Clinical Immunology, no. August: 1-12. https://doi.org/10.1111/all.15069.
Kuwahara, Akela, Ace E. Lewis, Coohleen Coombes, Fang Shiuan Leung, Michelle Percharde, and Jeffrey O. Bush. 2020. “Delineating the Early Transcriptional Specification of the Mammalian Trachea and Esophagus.” ELife 9: 1-23. https://doi.org/10.7554/eLife.55526.
Liu, Xuefeng, Ewa Krawczyk, Frank A Suprynowicz, Nancy Palechor-Ceron, Hang Yuan, Aleksandra Dakic, Vera Simic, et al. 2017. “Conditional Reprogramming and Long-Term Expansion of Normal and Tumor Cells from Human Biospecimens.” Nature Protocols 12 (2): 439-51. https://doi.org/10.1038/nprot.2016.174.
Madissoon, E., A. Wilbrey-Clark, R. J. Miragaia, K. Saeb-Parsy, K. T. Mahbubani, N. Georgakopoulos, P. Harding, et al. 2019. “ScRNA-Seq Assessment of the Human Lung, Spleen, and Esophagus Tissue Stability after Cold Preservation.” Genome Biology 21 (1): 1-16. https://doi.org/10.1186/s13059-019-1906-x.
McInnes, Leland, John Healy, and James Melville. 2018. “UMAP: Uniform Manifold Approximation and Projection for Dimension Reduction.” ArXiv.
Mou, Hongmei, Vladimir Vinarsky, Purushothama Rao Tata, Karissa Brazauskas, Soon H. Choi, Adrianne K. Crooke, Bing Zhang, et al. 2016. “Dual SMAD Signaling Inhibition Enables Long-Term Expansion of Diverse Epithelial Basal Cells.” Cell Stem Cell 19 (2): 217-31. https://doi.org/10.1016/j.stem.2016.05.012.
Pavel, Mariana, Maurizio Renna, So Jung Park, Fiona M Menzies, Thomas Ricketts, Jens Fullgrabe, Avraham Ashkenazi, et al. 2018. “Contact Inhibition Controls Cell Survival and Proliferation via YAP/TAZ-Autophagy Axis.” Nature Communications. https://doi.org/10.1038/s41467-018-05388-x.
Pepejoy, A, and S Fullerton. 2016. “Genomics Is Failing on Diversity.” Nature 538 (161-164). https://doi.org/https://doi.org/10.1038/538161a.
Piedrafita, Gabriel, Vasiliki Kostiou, Agnieszka Wabik, Bartomeu Colom, David Fernandez-Antoran, Albert Herms, Kasumi Murai, Benjamin A. Hall, and Philip H. Jones. 2020. “A Single-Progenitor Model as the Unifying Paradigm of Epidermal and Esophageal Epithelial Maintenance in Mice.” Nature Communications 11 (1): 1-15. https://doi.org/10.1038/s41467-020-15258-0.
Polanski, Krzysztof, Matthew D. Young, Zhichao Miao, Kerstin B. Meyer, Sarah A. Teichmann, and Jong Eun Park. 2020. “BBKNN: Fast Batch Alignment of Single Cell Transcriptomes.” Bioinformatics 36 (3): 964-65. https://doi.org/10.1093/bioinformatics/btz625.
Rankin, Scott A., Lu Han, Kyle W. McCracken, Alan P. Kenny, Christopher T. Anglin, Emily A. Grigg, Calyn M. Crawford, James M. Wells, John M. Shannon, and Aaron M. Zorn. 2016. “A Retinoic Acid-Hedgehog Cascade Coordinates Mesoderm-Inducing Signals and Endoderm Competence during Lung Specification.” Cell Reports 16 (1): 66-78. https://doi.org/10.1016/j.celrep.2016.05.060.
Rastogi, Amit, Srinivas Puli, Hashem B El-Serag, Ajay Bansal, Sachin Wani, and Prateek Sharma. 2008. “Incidence of Esophageal Adenocarcinoma in Patients with Barrett's Esophagus and High-Grade Dysplasia: A Meta-Analysis.” Gastrointestinal Endoscopy 67 (3): 394-98. https://doi.org/10.1016/j.gie.2007.07.019.
Rojas, A, P Araya, I Gonzalez, and E Morales. 2020. Tumor Microenvironments in Organs. Advances in Experimental Medicine and Biology. Vol. 1226. http://link.springer.com/10.1007/978-3-030-36214-0.
Rosekrans, Sanne L., Bart Baan, Vanesa Muncan, and Gijs R. Van Den Brink. 2015. “Esophageal Development and Epithelial Homeostasis.” American Journal of Physiology—Gastrointestinal and Liver Physiology 309 (4): G216-28. https://doi.org/10.1152/ajpgi.00088.2015.
Saraggi, Deborah, Matteo Fassan, Jan Bornschein, Fabio Farinati, Stefano Realdon, Nicola Valeri, and Massimo Rugge. 2016. “From Barrett Metaplasia to Esophageal Adenocarcinoma: The Molecular Background Histology and Histopathology,” 25-32. https://doi.org/10.14670/HH-11-659.
Sherley, J. L., P. B. Stadler, and J. Scott Stadler. 1995. “A Quantitative Method for the Analysis of Mammalian Cell Proliferation in Culture in Terms of Dividing and Non-dividing Cells.” Cell Proliferation 28 (3): 137-44. https://doi.org/10.1111/j.1365-2184.1995.tb00062.x.
Spechler, SJ, SK Jain, DA Tendler, and RA Parker. 2002. “Racial Differences in the Frequency of Symptoms and Complications of Gastro-Oesophageal Reflux Disease.” Alimentary Pharmacology & Therapeutics 16 (10): 1795-1800. https://doi.org/10.1046/j.0269-2813.2002.01351.x.
Spechler, Stuart J, Prateek Sharma, Rhonda F Souza, John M Inadomi, and Nicholas J Shaheen. 2011. “American Gastroenterological Association Medical Position Statement on the Management of Barrett's Esophagus.” Gastroenterology 140 (3): 1084-91. https://doi.org/10.1053/j.gastro.2011.01.030.
Spence, Jason R., Christopher N. Mayhew, Scott A. Rankin, Matthew F. Kuhar, Jefferson E. Vallance, Kathryn Tolle, Elizabeth E. Hoskins, et al. 2011. “Directed Differentiation of Human Pluripotent Stem Cells into Intestinal Tissue in Vitro.” Nature 470 (7332): 105-10. https://doi.org/10.1038/natureO9691.
Srinivasan, B, L Shuler, and James J Hickman. 2015. TEER Measurements in Cells. Journal of Laboratory Automation. Vol. 20. https://doi.org/10.1177/2211068214561025.TEER.
Suite, Queen St W. 2021. “Science Suite Inc—o/a BioRender,” 6337.
Suprynowicz, Frank A., Christopher M. Kamonjoh, Ewa Krawczyk, Seema Agarwal, Anton Wellstein, Fadeke A. Agboke, Sujata Choudhury, Xuefeng Liu, and Richard Schlegel. 2017. “Conditional Cell Reprogramming Involves Noncanonical β-Catenin Activation and MTOR-Mediated Inactivation of Akt.S.” PLoS ONE 12 (7): 1-23. https://doi.org/10.1371/journal.pone.0180897.
Then, Eric Omar, Michell Lopez, Saad Saleem, Vijay Gayam, Tagore Sunkara, Andrea Culliford, and Vinaya Gaduputi. 2020. “Esophageal Cancer: An Updated Surveillance Epidemiology and End Results Database Analysis.” World Journal of Oncology 11 (2): 55-64. https://doi.org/10.14740/WJON1254.
Thrift, Aaron P., and Hashem B. El-Serag. 2016. “Sex and Racial Disparity in Incidence of Esophageal Adenocarcinoma: Observations and Explanations.” Clinical Gastroenterology and Hepatology 14 (3): 330-32. https://doi.org/10.1016/j.cgh.2015.10.028.
Trisno, Stephen L., Katherine E. D. Philo, Kyle W. McCracken, Emily M. Catá, Sonya Ruiz-Torres, Scott A. Rankin, Lu Han, et al. 2018. “Esophageal Organoids from Human Pluripotent Stem Cells Delineate Sox2 Functions during Esophageal Specification.” Cell Stem Cell 23 (4): 501-515.e7. https://doi.org/10.1016/j.stem.2018.08.008.
Uhlen, M. 2005. “A Human Protein Atlas for Normal and Cancer Tissues Based on Antibody Proteomics.” Molecular & Cellular Proteomics 4 (12): 1920-32. https://doi.org/10.1074/mcp.M500279-MCP200.
Uhlen, M., L. Fagerberg, B. M. Hallstrom, C. Lindskog, P. Oksvold, A. Mardinoglu, A. Sivertsson, et al. 2015. “Tissue-Based Map of the Human Proteome.” Science 347 (6220): 1260419-1260419. https://doi.org/10.1126/science.1260419.
Vaughan, F. L., Kass L. L., and J. A Uzman. 1981. “Requirement of Hydrocortisone and Insulin for Extended Proliferation and Passage of Rat Keratinocytes.” In Vitro 17 (11): 941-46. https://doi.org/10.1007/BF02618418.
Weiler, Thomas, Irene Mikhail, Amit Singal, and Hemant Sharma. 2014. “Racial Differences in the Clinical Presentation of Pediatric Eosinophilic Esophagitis.” Journal of Allergy and Clinical Immunology: In Practice 2 (3): 320-25. https://doi.org/10.1016/j.jaip.2014.01.011.
Wolf, F. Alexander, Philipp Angerer, and Fabian J. Theis. 2017. “SCANPY: Large-Scale Single-Cell Gene Expression Data Analysis.” Genome Biology (2018) 19 (8): 2926-34. https://doi.org/10.1111/1462-2920.13787.
Yamamoto, Yusuke, Xia Wang, Denis Bertrand, Florian Kern, Ting Zhang, Marcin Duleba, Supriya Srivastava, et al. 2016. “Mutational Spectrum of Barrett's Stem Cells Suggests Paths to Initiation of a Precancerous Lesion.” Nature Communications 7: 1-10. https://doi.org/10.1038/ncomms10380.
Zhang, Yongchun, Dominique Bailey, Patrick Yang, Eugene Kim, and Jianwen Que. 2021. “The Development and Stem Cells of the Esophagus.” Development (Cambridge) 148 (6). https://doi.org/10.1242/dev.193839.
Zhang, Yongchun, Ming Jiang, Eugene Kim, Sijie Lin, Kuancan Liu, Xiaopeng Lan, and Jianwen Que. 2017. “Development and Stem Cells of the Esophagus.” Seminars in Cell and Developmental Biology 66: 25-35. https://doi.org/10.1016/j.semcdb.2016.12.008.
Zhang, Yongchun, Ying Yang, Ming Jiang, Sarah Xuelian Huang, Wanwei Zhang, Denise Al Alam, Soula Danopoulos, et al. 2018. “3D Modeling of Esophageal Development Using Human PSC-Derived Basal Progenitors Reveals a Critical Role for Notch Signaling.” Cell Stem Cell 23 (4): 516-529.e5. https://doi.org/10.1016/j.stem.2018.08.009.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method, comprising culturing obtained esophageal cells in vitro, wherein the culturing of the obtained esophageal cells results in expanded esophageal cells, wherein the culturing comprises seeding the obtained esophageal cells with a growth medium comprising hydrocortisone, epidermal growth factor (EGF), a rho-kinase inhibiting agent, a Wnt signaling pathway activating agent, a TGFβ inhibiting agent, and a BMP inhibiting agent.

2. The method of claim 1, wherein the growth medium comprises hydrocortisone, EGF, Y-276327, CHIR99021, A-8301, and noggin.

3. The method of claim 1, wherein the expanded esophageal cells are 2-dimensional esophageal cells and/or 3-dimensional esophageal cells.

4. The method of claim 1, wherein the amount of growth medium utilized in the culturing step is approximately 2 ml within a 9.8 cm² well, which results in generation of 2-dimensional expanded esophageal cells.

5. The method of claim 1, wherein the amount of growth medium utilized in the culturing step is approximately 4 ml within a 9.8 cm² well, which results in generation of 3-dimensional expanded esophageal cells.

6. The method of claim 1, the obtained esophageal cells are one or more of:
esophageal cells comprised within a biopsy sample from a subject (e.g., a human subject),
esophageal cells comprised within a surgical specimen sample from a subject (e.g., a human subject),
fetal esophageal cells,
adult esophageal cells,
esophageal stem cells, and
differentiated esophageal cells.

7. The method of claim 1, wherein the obtained esophageal cells are fetal esophageal cells.

8. The method of claim 1, wherein the obtained esophageal cells are adult esophageal cells.

9. The method of claim 1, wherein prior to the culturing step the esophageal cells are minced or enzymatically digested.

10. The method of claim 1, wherein the expanded esophageal cells possess esophageal stem cells and/or differentiated esophageal cells.

11. The method of claim 1, wherein the expanded esophageal cells are capable of being cryopreserved.

12. The method of claim 11, wherein the cryopreserved expanded esophageal cells can be thawed for purposes of generation of esophageal cell lines (e.g., 2-dimensional).

13. The method of claim 11, wherein the cryopreserved expanded esophageal cells can be thawed for purposes of generation of esophageal organoid tissue (e.g., 3-dimensional).

14. The method of claim 1, wherein the expanded esophageal cells are stratified esophageal epithelium cells.

15. The method of claim 1, wherein the expanded esophageal cells comprise 2-dimensional stratified esophageal epithelium.

16. The method of claim 1, wherein the expanded esophageal cells comprise 3-dimensional stratified esophageal epithelium (e.g., 3-dimensional esophageal organoid tissue).

17. The method of claim 1, wherein the culturing further comprises seeding the esophageal cells onto sub-lethally irradiated feeder cells and the growth media.

18. The method of claim 17, wherein the sub-lethally irradiated feeder cells are 3T3-J2i sub-lethally irradiated feeder cells.

19. The method of claim 1, wherein the culturing further comprises seeding the esophageal cells onto solubilized basement membrane matrix cells and the growth media.

20. The method of claim 19, wherein the solubilized basement membrane matrix cells are matrigel cells.

21. A composition comprising or consisting of expanded esophageal cells produced in vitro from the method of claim 1.

22. A kit comprising or consisting of expanded esophageal cells produced in vitro from the method of claim 1.
